(19) United States
(12) Patent Application Publication
(10) Pub. No.: US 2013/0123285 A1
(43) Pub. Date: May 16, 2013

(54) Compositions and Methods for Targeting A3G:RNA Complexes

(71) Applicants: Harold C. Smith, Rochester, NY (US); Kimberly Prohaska, Rochester, NY (US); William M. McDougall, Rochester, NY (US)

(72) Inventors: Harold C. Smith, Rochester, NY (US); Kimberly Prohaska, Rochester, NY (US); William M. McDougall, Rochester, NY (US)

(73) Assignee: UNIVERSITY OF ROCHESTER, ROCHESTER, NY (US)

(21) Appl. No.: 13/697,932

(22) PCT Filed: May 13, 2011
(86) PCT No.: PCT/US11/36430

§ 371 Date: Feb. 4, 2013

Related U.S. Application Data
(60) Provisional application No. 61/334,902, filed on May 14, 2010.

(51) Int. Cl. G01N 33/50; A61K 31/517; A61K 31/407

(52) U.S. Cl. CPC G01N 33/5008; A61K 31/407; A61K 31/517
USPC 514/266.22; 548/453; 514/413; 544/285; 506/9; 435/375

(57) ABSTRACT

The present invention provides an assay for screening any agent that modulates the ability of A3G to bind with RNA. The invention provides an agent identified by high throughput screening methods and methods of treatment using the identified agent as a means of inhibiting HIV infection and reducing the emergence of viral drug-resistance.

### BACKGROUND OF THE INVENTION

Human APOBEC3G or hA3G is a member of a family of cytidine deaminases that catalyze hydrolytic deamination of cytidine to uridine or deoxycytidine to deoxyuridine in the context of single stranded nucleic acids (Jarmuz, et al., 2002, Genomics. 79: 285-96; Wedekind, et al., 2003, Trends Genet. 19: 207-16). hA3G functions as an anti-lentiviral host factor (Sheehy, et al., 2002, Nature. 418: 646-650). Although deaminase-dependent and deaminase-independent hypotheses regarding the mechanism of hA3G antiviral activity have polarized research groups working in the field. The majority of the field believes that A3G has to be encapsidated with budding virions in order to exert its antiviral activity. Data presented here show that this need not be the case and demonstrate the antiviral and therapeutic potential of small molecules that can mobilize A3G from RNA-dependent high molecular mass aggregates. These results were unpredicable because they show that RNA-dependent inactivation of A3G is reversible both in vitro and in living cells and activates A3G host antiviral activity.

Several groups ascribed select amino acid residues within the C-terminal catalytic center as essential for antiviral ssDNA deaminase activity, whereas residues within and surrounding the pseudocatalytic center in the N-terminal half of hA3G are required for RNA binding, co-assembly with virions through Gag-dependent and Gag-independent interactions and mediate the ability of hA3G to block reverse transcription (Iwatani, et al., 2006, J Virol, 80: 5992-6002; Navarro, et al., 2005, Virology. 333: 374-86; Hakata, et al., 2006, J Biol Chem. 281:36624-31; Hache, et al., 2005, J Biol Chem. 280: 10920-4). Immunofluorescence studies demonstrated hA3G in a punctuate cytoplasmic distribution previously characterized as Processing bodies (P-bodies) (Wichroski, et al., 2006, PLoS Pathog. 2: e41) and stress granules (Stopak, et al., 2006, J Biol Chem. 282: 3539-46; Kozaket al., 2006, J Biol Chem. 281: 29105-19). Proteins characteristic of P-bodies or stress granules co-immunoprecipitate with hA3G, but fail to do so after ribonuclease digestion (Wichroski, et al., 2006, PLoS Pathog. 2: e41; Kozaket al., 2006, J Biol Chem. 281: 29105-19; Chiu, et al., 2006, Proc Natl Acad Sci USA. 103: 15588-93). In vitro hA3G binds nonspecifically to RNA or ssDNA (Kozaket al., 2006, J Biol Chem. 281: 29105-19; Chelico, et al., 2006, Nat Struct Mol Biol. 13: 392-9; Opi, et al., 2006, J Virol. 80: 4673-82) and therefore cellular RNA may nonspecifically associate hA3G with these cytoplasmic compartments.

Size exclusion chromatography and sucrose density sedimentation analyses showed that hA3G isolated from human cells was assembled as high molecular mass (HMM) complexes of 5-15 mDa (Chiu, et al., 2006, Proc Natl Acad Sci USA. 103: 15588-93; Chiu, et al., 2005, Nature. 435: 108-14; Kreisberg, et al., 2006, J Exp Med. 203: 865-70; Gallois-Montbrun, et al., 2007, J Virol 81, 2165-78). HMM complexes were dissociated to low molecular mass complexes (LMM) in vitro by digestion with ribonuclease. Interestingly, HMM complexes lacked deaminase activity when tested in vitro but were activated by ribonuclease treatment (Chelico, et al., 2006, Nat Struct Mol Biol. 13: 392-9; Opi, et al., 2006, J Virol. 80: 4673-82; Chiu, et al., 2005, Nature. 435: 108-14; Wedekind, et al., 2006, J Biol Chem. 281: 38122-6).

The recent collapse of HIV vaccine clinical trials underscores the need to renew efforts aimed at identifying novel drugs for HIV/AIDS therapy (Altman et al., 2008 Nature 452: 503). There exists a need in the field for novel HIV/AIDS therapy. The present invention satisfies this need as well as other needs regarding treatment of HIV infection.

### SUMMARY OF THE INVENTION

The present invention includes a method of identifying an agent that disrupts A3G:nucleic acid molecule interaction. In one embodiment, the method comprises contacting A3G in an A3G:nucleic acid molecule complex with a test agent under conditions that are effective for A3G:nucleic acid molecule complex formation, and detecting whether or not the test agent disrupts A3G:nucleic acid molecule interaction, wherein detection of disruption of A3G:nucleic acid molecule interaction identifies an agent that disrupts A3G:RNA nucleic acid molecule.

In one embodiment, the nucleic acid molecule is selected from the group consisting of ssDNA, RNA, and any combination thereof.

In another embodiment, the test agent that disrupts A3G:RNA interaction activates its ssDNA dC to dU deaminase activity as part of an inhibitor of lentiviral infectivity.

In another embodiment, the test agent that disrupts A3G:RNA interaction enables binding to ssDNA in lentiviral replications complexes as part of an inhibitor of lentiviral infectivity.

In one embodiment, the method of identifying an agent that disrupts A3G:nucleic acid molecule interaction is a high throughput method. In one embodiment, the high throughput method is Förster quenched resonance energy transfer (FqRET).

The present invention also includes an agent identified by a method of identifying an agent that disrupts A3G:nucleic acid molecule interaction.

The present invention also includes a method for inhibiting infectivity of a virus. In one embodiment, the method comprises contacting a cell with an antiviral-effective amount an agent identified by the methods of the invention.

In one embodiment, the virus is selected from the group consisting of HIV 1, HIV 2, hepatitis A, hepatitis B, hepatitis C, XMRV, and any combination thereof.

In another embodiment, the virus is associated with an RNA intermediate in the cytoplasm of cells.

In yet another embodiment, the virus is associated with DNA replication in the cytoplasm of cells.

In another embodiment, the virus comprises endogenous retroviral elements of the line, sine, and alu category.

In another embodiment, the virus is a foamy virus.

In one embodiment, the agent inhibits the interaction of A3G with RNA, thereby allowing the A3G to exhibit anti-viral activity.

In one embodiment, the agent is selected from the group consisting of Altanserin, Clonidine, and analogs thereof and having a related chemical scaffold (chemotype).

The present invention also includes a method for inhibiting A3G:RNA interaction in a cell. In one embodiment, the method comprises contacting A3G:RNA complex with an inhibitory-effective amount of an agent identified by the methods of the invention.

The present invention includes a method for treating or preventing HIV infection or AIDS in a patient. In one embodiment, the method comprises administering to a patient in need of such treatment or prevention a therapeutically effective amount of an agent identified according to the methods of the invention.

The invention also includes a method of attacking viral resistance. In one embodiment, the method comprises releasing RNA inactivation of A3G thereby activating A3G in a cell. In one embodiment, the A3G is not encapsidated in order to exert its antiviral activity. In another embodiment, the cell has not been infected by a virus and activation of A3G t preemptively inhibits viral replication.

In yet another embodiment, releasing RNA inactivation of A3G is accomplished by contacting a cell with an antiviral-effective amount of an agent identified according to the methods of the invention. In another embodiment, releasing RNA inactivation of A3G is accomplished by contacting a cell with an antiviral-effective amount of an agent selected from the group consisting of Altanserin, Clonidine, and analogs thereof and having a related chemical scaffold (chemotype).

The invention also includes a method of creating a reservoir of an active form of A3G in a cell prior to viral infection of the cell. In one embodiment, the method comprises disrupting A3G:RNA complex in the cell.

The invention also includes a method of reducing the emergence of viral drug-resistance in a cell. In one embodiment, the method comprises disrupting A3G:RNA complex in the cell.

### BRIEF DESCRIPTION OF THE DRAWINGS

For the purpose of illustrating the invention, there are depicted in the drawings certain embodiments of the invention. However, the invention is not limited to the precise arrangements and instrumentalities of the embodiments depicted in the drawings.

FIG. 1 is an image demonstrating that RNA displaces ssDNA from A3G.

FIG. 2 is an image depicting optimization of High Throughput Screening (HTS) assay conditions.

FIG. 3A is an image depicting a schematic of the assembly of complexes used in the FqRET HTS assay. FIG. 3B is image showing Coosnassie Blue stained gels of purified HMM and LMM (minus and plus RNase A digestion during protein purification).

FIG. 4, comprising FIG. 4A and FIG. 4B, is a series of images depicting protein-RNA complexes formed by Alexa647-A3G and QXL670-RNA. FIG. 4A depicts Alexa647 A3G was incubated for 1 hour with QXL670/32P-labeled RNA at the indicated temperatures. Reactions contained either 2.5- or 5-fold molar excess of RNA. Complex assembly was evaluated by EMSA and radiolabeled RNA detected using a Typhoon 9410 Phosphorimager. FIG. 4B depicts EMSA of 37° C. reactions were exposed to 647 nm light and scanned for fluorescence at 670 nm to reveal quenching in the A3G-RNA complexes.

FIG. 5 is an image depicting that RNase digestion demonstrates quenching requires A3G-RNA complexes.

FIG. 6 is an image depicting results from a library screen.

FIG. 7 is an image depicting four compounds that were selected from the library screen for further study.

FIG. 8 is an image depicting that ‘hit’ decrease A3G RNA binding as measured by electrophoretic mobility of HMM and LMM.

FIG. 9 is an image depicting that none of the ‘hits’ inhibited A3G deaminase activity (exemplified by clonidine and Altanserin).

FIG. 10 is an image depicting that the tested compounds did not inhibit A3G entry into viral particles.

FIG. 11 is an image depicting that A3G overexpressed in the infectivity reporter cell line (TMZ-bl) was aggregated as MDa, RNase-sensitive HMM.

FIG. 12 is an image depicting reactivation of A3G deaminase activity following treating of HMM in vitro with the test compounds.

FIG. 13 is a graph demonstrating that activation of cellular A3G reduces virus infectivity.

### DETAILED DESCRIPTION OF THE INVENTION

The present invention provides compositions and methods for targeting APOBEC3G (A3G) bound to a polynucleotide molecule. The present invention is based, at least in part, on the ability to disrupt complexes in which A3G is bound to RNA. Disrupting A3G:RNA complex serves to activate the host defense factor A3G by way of antagonizing the ability of RNA to bind to and aggregate A3G as HMM. Accordingly, the invention includes selectively targeting A3G binding to a polynucleotide molecule to activate host defense as an anti-viral therapy. Preferably, the polynucleotide molecule is RNA. The following description of the invention describes the invention in terms of disrupting or preventing formation of A3G:RNA complex. However, the invention should not be limited to A3G:RNA complexes. Rather, the invention includes disrupting or preventing any A3G:polynucleotide complex.

The present invention provides a screening assay to identify agents that disrupt A3G-RNA binding and the agents identified by the assay. For example, the agent includes, but is not limited, to Altanserin, Clonidine, and analogs thereof. However, the invention should not be limited to only these compounds, but should include any compound and analogs thereof that can be identified according to the screening methods of the invention.

In one embodiment, the invention provides a method for activating pre-existing A3G by disrupting A3G-RNA complexes. In other words, the invention includes a method that screens for compounds that have antiviral activity based on their ability to disrupt A3G-RNA complexes.

In another embodiment, the invention provides a method for activating pre-existing A3G in living cells by preventing formation of A3G-RNA complexes. In other words, the invention includes a method that screens for compounds that have antiviral activity based on their ability to prevent formation of A3G-RNA complexes.

Inhibiting or reducing the interaction between A3G and RNA allows A3G to exist in an active form, for example, switching on the deaminase-dependent and -independent antiviral activities of A3G that inhibit HIV replication. In one instance, if a cell that is producing virus is treated with an agent that inhibits A3G and RNA, the virus that is being produced by the cell is inactivated and thus is unable (or exhibits a reduced capacity) to carry out future rounds of infection. In this manner, infectivity of the virus is inhibited by the compounds identified by the screening methods of the invention.

In one embodiment, the invention provides compositions and method to relieve RNA inactivation of A3G as HMM. In some instances, RNA inactivation of A3G is reversible and once A3G is activated, A3G can exert antiviral activity against incoming virus. In some instances, compositions of the invention target A3G:RNA complexes in a nonspecific manner and are able to inhibit viral replication and integration. Therefore, in some instances, the compositions of the invention do not depend exclusively on A3G encapsidation for therapeutic efficacy. Thus, the invention offers a novel opportunity for attacking viral resistance.

In one embodiment, the invention provides a method of activating cellular A3G in a cell as a preemptive measure to inhibit viral infection, replication and integration into the cells chromosomal DNA. That is, in one embodiment, the invention provides a method to create a reservoir of an active form of A3G prior to viral infection.

The methods disclosed herein allow for rapid screening of agents for their ability to inhibit interaction between A3G and RNA, which agents provide a therapeutic benefit, including, but not limited to, treating viral infection, while reducing the risk of cell toxicity that might otherwise arise form other types of anti-viral therapy. Preferably, the viral infection is HIV.

### DEFINITIONS

As used herein, each of the following terms has the meaning associated with it in this section.

The articles “a” and “an” are used herein to refer to one or to more than one (e.g., to at least one) of the grammatical object of the article. By way of example, “an element” means one element or more than one element.

The term “binding” refers to a direct association between at least two molecules, due to, for example, covalent, electrostatic, hydrophobic, ionic and/or hydrogen-bond interactions under physiological conditions.

As used herein, the term “fragment,” as applied to a nucleic acid, refers to a subsequence of a larger nucleic acid. A “fragment” of a nucleic acid can be at least about 20 nucleotides in length; for example, at least about 50 nucleotides to about 100 nucleotides; preferably at least about 100 to about 500 nucleotides, more preferably at least about 500 to about 1000 nucleotides, even more preferably at least about 1000 nucleotides to about 1500 nucleotides; particularly, preferably at least about 1500 nucleotides to about 2500 nucleotides; most preferably at least about 2500 nucleotides.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting there from. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

“Expression vector” refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, such as cosmids, plasmids (e.g., naked or contained in liposomes) and viruses (e.g., retroviruses, lentiviruses, adenoviruses, and adeno-associated viruses) that incorporate the recombinant polynucleotide.

As used herein, the term “gene” refers to an element or combination of elements that are capable of being expressed in a cell, either alone or in combination with other elements. In general, a gene comprises (from the 5′ to the 3′ end): (1) a promoter region, which includes a 5′ nontranslated leader sequence capable of functioning in any cell such as a prokaryotic cell, a virus, or a eukaryotic cell (including transgenic mammals); (2) a structural gene or polynucleotide sequence, which codes for the desired protein; and (3) a 3′ nontranslated region, which typically causes the termination of transcription and the polyadenylation of the 3′ region of the RNA sequence. Each of these elements is operatively linked by sequential attachment to the adjacent element. A gene comprising the above elements is inserted by standard recombinant DNA methods into any expression vector.

As used herein, “gene products” include any product that is produced in the course of the transcription, reverse-transcription, polymerization, translation, post-translation and/or expression of a gene. Gene products include, but are not limited to, proteins, polypeptides, peptides, peptide fragments, or polynucleotide molecules.

“Homologous” as used herein, refers to the subunit sequence similarity between two polymeric molecules, e.g., between two nucleic acid molecules, e.g., two DNA molecules or two RNA molecules, or between two polypeptide molecules. When a subunit position in both of the two molecules is occupied by the same monomeric subunit, e.g., if a position in each of two DNA molecules is occupied by adenine, then they are homologous at that position. The homology between two sequences is a direct function of the number of matching or homologous positions, e.g., if half (e.g., five positions in a polymer ten subunits in length) of the positions in two compound sequences are homologous then the two sequences are 50% homologous, if 90% of the positions, e.g., 9 of 10, are matched or homologous, the two sequences share 90% homology. By way of example, the DNA sequences 5′ATTGCC3′ and 5′TATGGC3′ share 50% homology.

As used herein, “homology” is used synonymously with “identity.”

The term “isolated nucleic acid molecule” includes nucleic acid molecules which are separated from other nucleic acid molecules which are present in the natural source of the nucleic acid. For example, with regards to genomic DNA, the term “isolated” includes nucleic acid molecules which are separated from the chromosome with which the genomic DNA is naturally associated. Preferably, an “isolated” nucleic acid is free of sequences which naturally flank the nucleic acid (i.e., sequences located at the 5′ and 3′ ends of the nucleic acid) in the genomic DNA of the organism from which the nucleic acid is derived. Moreover, an “isolated” nucleic acid molecule can be substantially free of other cellular material, or culture medium when produced by recombinant techniques, or substantially free of chemical precursors or other chemicals when chemically synthesized.

The term “lentivirus” as used herein may be any of a variety of members of this genus of viruses. The lentivirus may be, e.g., one that infects a mammal, such as a sheep, goat, horse, cow or primate, including human. Typical such viruses include, e.g., Vizna virus (which infects sheep); simian immunodeficiency virus (SIV), bovine immunodeficiency virus (BIV), chimeric simian/human immunodeficiency virus (SHIV), feline immunodeficiency virus (FIV) and human immunodeficiency virus (HIV). “HIV,” as used herein, refers to both HIV-1 and HIV-2. Much of the discussion herein is directed to HIV or HIV-1; however, it is to be understood that other suitable lentiviruses are also included.

The term “mammal” as used herein refers to any non-human mammal. Such mammals are, for example, rodents, non-human primates, sheep, dogs, cows, and pigs. The preferred non-human mammals are selected from the rodent family including rat and mouse, more preferably mouse. The preferred mammal is a human.

A “nucleic acid molecule” is intended generally to include DNA molecules (e.g., cDNA or genomic DNA) and RNA molecules (e.g., mRNA) and analogs of the DNA or RNA generated using nucleotide analogs. The nucleic acid molecule can be single-stranded or double-stranded, but preferably is double-stranded DNA.

The term “operably linked” refers to functional linkage between a regulatory sequence and a heterologous nucleic acid sequence resulting in expression of the latter. For example, a first nucleic acid sequence is operably linked with a second nucleic acid sequence when the first nucleic acid sequence is placed in a functional relationship with the second nucleic acid sequence. For instance, a promoter is operably linked to a coding sequence if the promoter affects the transcription or expression of the coding sequence. Generally, operably linked DNA sequences are contiguous and, where necessary to join two protein coding regions, in the same reading frame.

As used herein, the terms “peptide,” “polypeptide,” and “protein” are used interchangeably, and refer to a compound comprised of amino acid residues covalently linked by peptide bonds. A protein or peptide must contain at least two amino acids, and no limitation is placed on the maximum number of amino acids which can comprise a protein's or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptide, homodimers, heterodimers, variants of polypeptides, modified polypeptides, derivatives, analogs, fusion proteins, among others. The polypeptides include natural peptides, recombinant peptides, synthetic peptides, or a combination thereof.

As used herein, “polynucleotide” includes cDNA, RNA, DNA/RNA hybrid, line, sine and alu elements, endogenous retroviral elements, retroviruses, anti-sense RNA, ribozyme, siRNA, genomic DNA, synthetic forms, and mixed polymers, both sense and antisense strands, and may be chemically or biochemically modified to contain non-natural or derivatized, synthetic, or semi-synthetic nucleotide bases. Also, included within the scope of the invention are alterations of a wild type or synthetic gene, including but not limited to deletion, insertion, substitution of one or more nucleotides, or fusion to other polynucleotide sequences, provided that such changes in the primary sequence of the gene do not alter the expressed peptide ability to elicit passive immunity.

“Pharmaceutically acceptable” means physiologically tolerable, for either human or veterinary applications. In addition, “pharmaceutically acceptable” is meant a material that is not biologically or otherwise undesirable, i.e., the material may be administered to a subject without causing any undesirable biological effects or interacting in a deleterious manner with any of the other components of the pharmaceutical composition in which it is contained. Essentially, the pharmaceutically acceptable material is nontoxic to the recipient. The carrier would naturally be selected to minimize any degradation of the active ingredient and to minimize any adverse side effects in the subject, as would be well known to one of skill in the art. For a discussion of pharmaceutically acceptable carriers and other components of pharmaceutical compositions, see, e.g., Remington's Pharmaceutical Sciences, 18th ed., Mack Publishing Company, 1990.

As used herein, “pharmaceutical compositions” include formulations for human and veterinary use.

As used herein, the terms “prevent,” “preventing,” “prevention,” “prophylactic treatment” and the like refer to reducing the probability of developing a disorder or condition in a subject, who does not have, but is at risk of or susceptible to developing a disorder or condition.

A “recombinant nucleic acid” is any nucleic acid that has been placed adjacent to another nucleic acid by recombinant DNA techniques. A “recombined nucleic acid” also includes any nucleic acid that has been placed next to a second nucleic acid by a laboratory genetic technique such as, for example, transformation and integration, transposon hopping or viral insertion. In general, a recombined nucleic acid is not naturally located adjacent to the second nucleic acid.

The term “recombinant protein” refers to a protein of the present invention which is produced by recombinant DNA techniques, wherein generally DNA encoding the expressed protein is inserted into a suitable expression vector which is in turn used to transform a host cell to produce the heterologous protein. Moreover, the phrase “derived from”, with respect to a recombinant gene encoding the recombinant protein is meant to include within the meaning of “recombinant protein” those proteins having an amino acid sequence of a native protein, or an amino acid sequence similar thereto which is generated by mutations including substitutions and deletions of a naturally occurring protein.

“Test agents” or otherwise “test compounds” as used herein refers to an agent or compound that is to be screened in one or more of the assays described herein. Test agents include compounds of a variety of general types including, but not limited to, small organic molecules, known pharmaceuticals, polypeptides; carbohydrates such as oligosaccharides and polysaccharides; polynucleotides; lipids or phospholipids; fatty acids; steroids; or amino acid analogs. Test agents can be obtained from libraries, such as natural product libraries and combinatorial libraries. In addition, methods of automating assays are known that permit screening of several thousands of compounds in a short period.

As used herein, the terms “treat,” “treating,” “treatment,” and the like refer to reducing or ameliorating a disorder and/or symptoms associated therewith. It will be appreciated that, although not precluded, treating a disorder or condition does not require that the disorder, condition or symptoms associated therewith be completely eliminated.

“Variant” as the term is used herein, is a nucleic acid sequence or a peptide sequence that differs in sequence from a reference nucleic acid sequence or peptide sequence respectively, but retains essential properties of the reference molecule. Changes in the sequence of a nucleic acid variant may not alter the amino acid sequence of a peptide encoded by the reference nucleic acid, or may result in amino acid substitutions, additions, deletions, fusions and truncations. Changes in the sequence of peptide variants are typically limited or conservative, so that the sequences of the reference peptide and the variant are closely similar overall and, in many regions, identical. A variant and reference peptide can differ in amino acid sequence by one or more substitutions, additions, deletions in any combination. A variant of a nucleic acid or peptide can be a naturally occurring such as an allelic variant, or can be a variant that is not known to occur naturally. Non-naturally occurring variants of nucleic acids and peptides may be made by mutagenesis techniques or by direct synthesis.

“Viral infectivity” as that term is used herein means any of the infection of a cell, the replication of a virus therein, and the production of progeny virions therefrom.

A “virion” is a complete viral particle; nucleic acid and capsid, further including and a lipid envelope in the case of some viruses.

### DESCRIPTION

The present invention is based on the discovery that selectively targeting A3G binding to RNA to activate the host defense can be used as an effective anti-viral therapy in which encapsidation is not required for A3G antiviral mechanism of antiviral action. In one embodiment, the present invention provides a method of overcoming HIV resistance to host defense mechanisms by activating A3G with agents that dissociate A3G-RNA complexes.

Accordingly, the invention includes a screening method that disrupt A3G:RNA complex and agents identified by the screening method that is designed to be bias and based on A3G complexes with RNA. The identified agents are considered antiviral compounds because they dissociate RNA from A3G and thereby ‘switch on’ the antiviral property of A3G. Consequently, the host-defense factors are positioned to interact with viral replication complexes and thereby block viral infectivity.

The assays described here are unique and are an enabling technology for the HIV/AIDS drug discovery industry because they are based on two discoveries. One, that RNA binding to A3G and inactivation of A3G are reversible. Two, RNA binding to A3G displaces and inhibits single stranded DNA substrates (such as ssDNA formed during reverse transcription during HIV replication) binding to A3G as the basis for why RNA binding to A3G inhibits A3G host antiviral activity.

### Method of Screening

The current invention relates to a method of screening for a compound that modulates or regulates the formation of an RNA-protein complex formed in vivo or in vitro. Preferably, the RNA-protein complex is RNA-A3G. In one embodiment, the screening method comprises contacting an A3G:RNA complex with a test compound under conditions that are effective for A3G:RNA complex formation and detecting whether or not the test agent disrupts A3G:RNA, wherein detection of disruption of A3G:RNA interaction identifies an agent that disrupts A3G:RNA interaction.

Other methods, as well as variation of the methods disclosed herein will be apparent from the description of this invention. For example, the test compound may be either fixed or increased, a plurality of compounds or proteins may be tested at a single time. “Modulation”, “modulates”, and “modulating” can refer to enhanced formation of the RNA-protein complex, a decrease in formation of the RNA-protein complex, a change in the type or kind of the RNA-protein complex or a complete inhibition of formation of the RNA-protein complex. Suitable compounds that may be used include but are not limited to proteins, nucleic acids, small molecules, hormones, antibodies, peptides, antigens, cytolines, growth factors, pharmacological agents including chemotherapeutics, carcinogenics, or other cells (i.e. cell-cell contacts). Screening assays can also be used to map binding sites on RNA or protein. For example, tag sequences encoding for RNA tags can be mutated (deletions, substitutions, additions) and then used in screening assays to determine the consequences of the mutations.

The invention relates to a method for screening test agents, test compounds or proteins for their ability to modulate or regulate an RNA-protein complex. By performing the methods of the present invention for purifying RNA-protein complexes formed in vitro or in vivo and observing a difference, if any, between the RNA-protein complexes purified in the presence and absence of the test, agents, test compounds or proteins, wherein a difference indicates that the test agents, test compounds or proteins modulate the RNA-protein complex.

One aspect of the invention is a method for identifying an agent (e.g. screening putative agents for one or more that elicits the desired activity) that inhibits the infectivity of a lentivirus. Typical such lentiviruses include, e.g., SW, SHIV and/or HIV. The method takes advantage of the successful production of large-scale amounts of recombinant A3G. This allows for assays that detect an agent that is capable of interfering with the interaction between A3G and RNA. An agent that interferes with A3G:RNA complex would be expected to inhibit infectivity of a lentivirus. Furthermore, such an agent would not be expected to interfere with the function of cellular proteins and thus would be expected to elicit few, if any, side effects as a result of disruption of A3G:RNA complex.

The method comprises: (a) contacting a putative inhibitory agent with a mixture comprising RNA and A3G under conditions that are effective for A3G:RNA complex formation; and (b) detecting whether the presence of the agent decreases the level of A3G:RNA complex formation. In some instances, the agent binds to A3G and thereby inhibits A3G:RNA complex formation. In another instance, the agent binds to RNA and thereby inhibits A3G:RNA complex formation. Any of a variety of conventional procedures can be used to early out such an assay.

In another embodiment, the method comprises: (a) contacting a putative inhibitory agent with a mixture comprising A3G:RNA complex under conditions that are effective for maintaining A3G:RNA complex; and (b) detecting whether the presence of the agent disrupts the A3G:RNA complex. In some instances, the agent binds to A3G and thereby disrupts A3G:RNA complex. In another instance, the agent binds to RNA and thereby disrupts A3G:RNA complex formation. Any of a variety of conventional procedures can be used to carry out such an assay.

The invention encompasses methods to identify a compound that inhibits the interaction between A3G and a nucleic acid molecule. In one embodiment, the nucleic molecule is RNA. In another embodiment, the nucleic acid molecule is ssDNA. However, the invention should not be limited to any particular type of nucleic acid molecule. Rather, a skilled artisan when armed with the present disclosure would understand that targeting any A3G:nucleic acid molecule complex is encompassed in the invention. As a non-limiting example, the disclosure refers to A3G:RNA complexes. Accordingly, In one embodiment, the invention provides an assay for determining the binding between A3G with RNA. The method includes contacting recombinant A3G and RNA in the presence of a candidate compound. Detecting inhibition or a reduced amount of A3G:RNA complex in the presence of the candidate compound compared to the amount of A3G:RNA complex in the absence of the candidate compound is an indication that the candidate compound is an inhibitor of A3G:RNA interaction.

Based on the disclosure presented herein, the screening method of the invention is applicable to a robust Förster quenched resonance energy transfer (FqRET) assay for high-throughput compound library screening in microtiter plates. The assay is based on selective placement of chromoproteins or chromophores that allow reporting on complex formation between the A3G and RNA in vitro. For example, an appropriately positioned FRET donor and FRET quencher will results in a “dark” signal when the quaternary complex is formed between A3G and RNA. However, the screening methods should not be limited solely to the assays disclosed herein. Rather, the recombinant proteins and RNA of the invention can be used in any assay, including other high-throughput screening assays, that are applicable for screening agents that regulate the binding between to RNA and protein. Thus, the invention encompasses the use of the recombinant proteins and RNAs of the invention in any assay that is useful for detecting an agent that interferes with protein-RNA interaction.

The skilled artisan would also appreciate, in view of the disclosure provided herein, that standard binding assays known in the art, or those to be developed in the future, can be used to assess the binding of A3G and RNA of the invention in the presence or absence of the test compound to identify a useful compound. Thus, the invention includes any compound identified using this method.

The screening method includes contacting a mixture comprising recombinant A3G and RNA with a test compound and detecting the presence of the A3G:RNA complex, where a decrease in the level of A3G:RNA complex compared to the amount in the absence of the test compound or a control indicates that the test compound is able to inhibit the binding between A3G and RNA. In certain embodiments, the control is the same assay performed with the test compound at a different concentration (e.g. a lower concentration), or in the absence of the test agent, etc.

Without wishing to be bound by any particular theory, it is believed that the A3G:RNA complex contains a ceiling level of complex formation because the presence the A3G and RNA has a propensity to bind with each other in the absence of a known control inhibitor. The activity of a test compound can be measured by determining whether the test compound can decrease the level of A3G:RNA complex formation.

Determining the ability of the test compound to interfere with the formation of the A3G:RNA complex, can be accomplished, for example, by coupling the A3G protein or RNA with a tag, radioisotope, or enzymatic label such that the A3G:RNA complex can be measured by detecting the labeled component in the complex. For example, a component of the complex (e.g., A3G or RNA) can be labeled with ³²P, ¹²⁵I, ³⁵S, ¹⁴C, or ³H, either directly or indirectly, and the radioisotope detected by direct counting of radioemission or by scintillation counting. Alternatively, a component of the complex can be enzymatically labeled with, for example, horseradish peroxidase, alkaline phosphatase, or luciferase, and the enzymatic label is then detected by determination of conversion of an appropriate substrate to product.

Determining the ability of the test compound to interfere with the A3G:RNA complex can also be accomplished using technology such as real-time Biomolecular Interaction Analysis (BIA) as described in Sjolander et al., 1991, Anal. Chem. 63:2338-2345 and Szabo et al., 1995, Curr. Opin. Struct. Biol. 5:699-705. BIA is a technology for studying biospecific interactions in real time, without labeling any of the interactants (e.g., BIAcore, BIAcore International AB, Uppsala, Sweden). Changes in the optical phenomenon of surface plasmon resonance (SPR) can be used as an indication of real-time reactions between biological molecules.

In more than one embodiment of the methods of the present invention, it may be desirable to immobilize either A3G or RNA to facilitate separation of complexed from uncomplexed forms of one or both of the molecules, as well as to accommodate automation of the assay. The effect of a test compound on the A3G:RNA complex, can be accomplished using any vessel suitable for containing the reactants. Examples of such vessels include microliter plates, test tubes, and micro-centrifuge tubes. In one embodiment, a fusion protein can be provided which adds a domain that allows one or both of the proteins to be bound to a matrix. For example, glutathione-S-transferase/target fusion proteins can be adsorbed onto glutathione sepharose beads (Sigma Chemical, St. Louis, Mo.) or glutathione-derivatized micrometer plates, which are then combined with the other corresponding component of the A3G:RNA complex in the presence of the test compound. The mixture is incubated under conditions conducive to complex formation (e.g., at physiological conditions for salt and pH). Following incubation, the beads or microtiter plate wells are washed to remove any unbound material, the matrix is immobilized in the case of beads, and the formation of the complex is determined either directly or indirectly, for example, as described above.

The test compounds can be obtained using any of the numerous approaches in combinatorial library methods known in the art, including: biological libraries; spatially addressable parallel solid phase or solution phase libraries; synthetic library methods requiring deconvolution; the “one-bead one-compound” library method; and synthetic library methods using affinity chromatography selection. The biological library approach is limited to peptide libraries, while the other four approaches are applicable to peptide, non-peptide oligomer or small molecule libraries of compounds (Lam et al., 1997, Anticancer Drug Des. 12:45).

Examples of methods for the synthesis of molecular libraries can be found in the art, for example, in: DeWitt et al., 1993, Proc. Natl. Acad. USA 90:6909; Erb et al., 1994, Proc. Natl. Acad. Sci. USA 91:11422; Zuckermann et al., 1994, J. Med. Chem. 37:2678; Cho et al., 1993, Science 261:1303; Carrell et al., 1994, Angew. Chem. Int. Ed. Engl. 33:2059; Carell et al., 1994, Angew. Chem. Int. Ed. Engl. 33:2061; and Gallop et al., 1994, J. Med. Chem. 37:1233.

Libraries of compounds may be presented in solution (e.g., Houghten, 1992, Biotechniques 13:412-421), or on beads (Lam, 1991, Nature 354:82-84), chips (Fodor, 1993, Nature 364:555-556), bacteria (Ladner U.S. Pat. No. 5,223,409), spores (Ladner U.S. Pat. No. '409), plasmids (Cull et al., 1992, Proc. Natl. Acad. Sci, USA 89:1865-1869) or on phage (Scott and Smith, 1990, Science 249:386-390; Devlin, 1990, Science 249:404-406; Cwirla et al., 1990, Proc. Natl. Acad. Sci. USA 87:6378-6382; Felici, 1991, J. Mol. Biol. 222:301-310; and Ladner supra).

In situations where “high-throughput” modalities are preferred, it is typical to that new chemical entities with useful properties are generated by identifying a chemical compound (called a “lead compound”) with some desirable property or activity, creating variants of the lead compound, and evaluating the property and activity of those variant compounds. The current trend is to shorten the time scale for all aspects of drug discovery.

In one embodiment, high throughput screening methods involve providing a library containing a large number of compounds (candidate compounds) potentially having the desired activity. Such “combinatorial chemical libraries” are then screened in one or more assays, as described herein, to identify those library members (particular chemical species or subclasses) that display a desired characteristic activity. The compounds thus identified can serve as conventional “lead compounds” or can themselves be used as potential or actual therapeutics.

### Methods of Treatment

In one embodiment, the present invention provides methods of treating a disease, disorder, or condition associated with a viral infection. Preferably, the viral infection is HIV. The method comprises administering to a subject, such as a mammal, preferably a human, a therapeutically effective amount of a pharmaceutical composition that inhibits the interaction between A3G and RNA.

The invention includes compounds identified using the screening methods discussed elsewhere herein. Such a compound can be used as a therapeutic to treat an HIV infection or otherwise a disorder associated with the inability to dissociate A3G:RNA complexes.

The ability for a compound to inhibit the interaction between A3G and RNA can provide a therapeutic to protect or otherwise prevent viral infection, for example HIV infection.

Thus, the invention includes pharmaceutical compositions. Pharmaceutically acceptable carriers that are useful include, but are not limited to, glycerol, water, saline, ethanol and other pharmaceutically acceptable salt solutions such as phosphates and salts of organic acids. Examples of these and other pharmaceutically acceptable carriers are described in Remington's Pharmaceutical Sciences (1991, Mack Publication Co., New Jersey), the disclosure of which is incorporated by reference as if set forth in its entirety herein.

The pharmaceutical compositions may be prepared, packaged, or sold in the form of a sterile injectable aqueous or oily suspension or solution. This suspension or solution may be formulated according to the known art, and may comprise, in addition to the active ingredient, additional ingredients such as the dispersing agents, wetting agents, or suspending agents described herein. Such sterile injectable formulations may be prepared using a non-toxic peritoneally-acceptable diluent or solvent, such as water or 1,3-butane diol, for example. Other acceptable diluents and solvents include, but are not limited to, Ringer's solution, isotonic sodium chloride solution, and fixed oils such as synthetic mono- or di-glycerides.

Pharmaceutical compositions that are useful in the methods of the invention may be administered, prepared, packaged, and/or sold in formulations suitable for oral, rectal, vaginal, peritoneal, topical, pulmonary, intranasal, buccal, ophthalmic, or another route of administration. Other contemplated formulations include projected nanoparticles, liposomal preparations, resealed erythrocytes containing the active ingredient, and immunologically-based formulations.

The compositions of the invention may be administered via numerous routes, including, but not limited to, oral, rectal, vaginal, peritoneal, topical, pulmonary, intranasal, buccal, or ophthalmic administration routes. The route(s) of administration will be readily apparent to the skilled artisan and will depend upon any number of factors including the type and severity of the disease being treated, the type and age of the veterinary or human patient being treated, and the like.

As used herein, “peritoneal administration” of a pharmaceutical composition includes any route of administration characterized by physical breaching of a tissue of a subject and administration of the pharmaceutical composition through the breach in the tissue. Peritoneal administration thus includes, but is not limited to, administration of a pharmaceutical composition by injection of the composition, by application of the composition through a surgical incision, by application of the composition through a tissue-penetrating non-surgical wound, and the like. In particular, peritoneal administration is contemplated to include, but is not limited to, subcutaneous, intraperitoneal, intramuscular, intrasternal injection, and kidney dialytic infusion techniques.

A pharmaceutical composition can consist of the active ingredient alone, in a form suitable for administration to a subject, or the pharmaceutical composition may comprise the active ingredient and one or more pharmaceutically acceptable carriers, one or more additional ingredients, or some combination of these. The active ingredient may be present in the pharmaceutical composition in the form of a physiologically acceptable ester or salt, such as in combination with a physiologically acceptable cation or anion, as is well known in the art.

The formulations of the pharmaceutical compositions described herein may be prepared by any method known or hereafter developed in the art of pharmacology. In general, such preparatory methods include the step of bringing the active ingredient into association with a carrier or one or more other accessory ingredients, and then, if necessary or desirable, shaping or packaging the product into a desired single- or multi-dose unit.

Although the descriptions of pharmaceutical compositions provided herein are principally directed to pharmaceutical compositions that are suitable for ethical administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and perform such modification with merely ordinary, if any, experimentation. Subjects to which administration of the pharmaceutical compositions of the invention is contemplated include, but are not limited to, humans and other primates, mammals including commercially relevant mammals such as cattle, pigs, horses, sheep, cats, and dogs.

Controlled- or sustained-release formulations of a pharmaceutical composition of the invention may be made using conventional technology.

Formulations of a pharmaceutical composition suitable for peritoneal administration comprise the active ingredient combined with a pharmaceutically acceptable carrier, such as sterile water or sterile isotonic saline. Such formulations may be prepared, packaged, or sold in a form suitable for bolus administration or for continuous administration. Injectable formulations may be prepared, packaged, or sold in unit dosage form, such as in ampules or in multi-dose containers containing a preservative. Formulations for peritoneal administration include, but are not limited to, suspensions, solutions, emulsions in oily or aqueous vehicles, pastes, and implantable sustained-release or biodegradable formulations. Such formulations may further comprise one or more additional ingredients including, but not limited to, suspending, stabilizing, or dispersing agents. In one embodiment of a formulation for peritoneal administration, the active ingredient is provided in dry (i.e., powder or granular) form for reconstitution with a suitable vehicle (e.g., sterile pyrogen-free water) prior to peritoneal administration of the reconstituted composition.

The pharmaceutical compositions may be prepared, packaged, or sold in the form of a sterile injectable aqueous or oily suspension or solution. This suspension or solution may be formulated according to the known art, and may comprise, in addition to the active ingredient, additional ingredients such as the dispersing agents, wetting agents, or suspending agents described herein. Such sterile injectable formulations may be prepared using a non-toxic peritoneally-acceptable diluent or solvent, such as water or 1,3-butane diol, for example. Other acceptable diluents and solvents include, but are not limited to, Ringer's solution, isotonic sodium chloride solution, and fixed oils such as synthetic mono- or di-glycerides. Other parentally-administrable formulations which are useful include those which comprise the active ingredient in microcrystalline form, in a liposomal preparation, or as a component of a biodegradable polymer systems. Compositions for sustained release or implantation may comprise pharmaceutically acceptable polymeric or hydrophobic materials such as an emulsion, an ion exchange resin, a sparingly soluble polymer, or a sparingly soluble salt.

Formulations suitable for topical administration include, but are not limited to, liquid or semi-liquid preparations such as liniments, lotions, oil-in-water or water-in-oil emulsions such as creams, ointments or pastes, and solutions or suspensions. Topically-administrable formulations may for example, comprise from about 1% to about 10% (w/w) active ingredient, although the concentration of the active ingredient may be as high as the solubility limit of the active ingredient in the solvent. Formulations for topical administration may further comprise one or more of the additional ingredients described herein.

Typically, dosages of the compound of the invention which may be administered to an animal, preferably a human, will vary depending upon any number of factors, including but not limited to, the type of animal and type of disease state being treated, the age of the animal and the route of administration.

The compound can be administered to an animal as frequently as several times daily, or it may be administered less frequently, such as once a day, once a week, once every two weeks, once a month, or even less frequently, such as once every several months or even once a year or less. The frequency of the dose will be readily apparent to the skilled artisan and will depend upon any number of factors, such as, but not limited to, the type and severity of the disease being treated, the type and age of the animal, and the like. Preferably, the compound is, but need not be, administered as a bolus injection that provides lasting effects for at least one day following injection. The bolus injection can be provided intraperitoneally.

### EXAMPLES

The invention is now described with reference to the following Examples. These Examples are provided for the purpose of illustration only, and the invention is not limited to these Examples, but rather encompasses all variations which are evident as a result of the teachings provided herein.

The preponderance of hA3G in activated CD4+ cells (that support HIV replication) is recovered in the HMM form, whereas that in uninfected resting CD4+ cells (that are resistant to HIV infection) is LMM (Chiu, et al., 2005, Nature, 435: 108-14; Kreisberg, et al., 2006, J Exp Med. 203: 865-70; Chiu, et al., 2006, J Biol Chem. 281: 8309-12; Cullen, 2006, J Virol. 80: 1067-76). HIV preferentially infected cells in which most of the hA3G was in HMM complexes in experimentally mixed T-lymphocyte cell populations (Kreisberg, et al., 2006, J Exp Med. 203: 865-70). Increased recovery of hA3G in HMM also was observed during maturation of monocytes to macrophages, a differentiation process associated with increased permissiveness to HIV infection, but also a reduction in the abundance of total cellular hA3G (Stopak, et al., 2006, J Biol Chem. 282: 3539-46; Chiu, et al., 2005, Nature. 435: 108-14; Peng et al., 2006, J Exp Med. 203: 41-6). Maturation of dendritic cells (DC) was associated with increased expression of hA3G mRNA and protein. However, in contrast to PBMC, mature DC became less permissive to R5 trophic HIV. An increased percentage of the total cellular hA3G in mature DC (differentiated in vitro with poly(I:C)/TNF-alpha) was in LMM complexes compared to immature DC (Stopak, et al., 2006, J Biol Chem. 282: 3539-46). Mature DC therefore either lacked means to form HMM or actively inhibited hA3G interactions with cellular RNA. The antiviral activity of A3G arises from its ability to physically block progression of the viral replication machinery as well as to bind to nascent proviral DNA and catalyze multiple mutations through dC to dU transitions (deamination). These activities are absent when activated T cells return to their resting state (Santoni de Sio et al., 2009 PLoS One 4: e6571) because A3G remains sequestered in high molecular mass (HMM) aggregates. HMM complexes may be composed of multiple (4 to >20) inactivated A3G subunits tethered together through nonspecific binding of A3G to cellular RNAs (Chiu et al., 2005 Nature 435: 108-114; Gallois-Montbrun et al., 2007 J Virol, 81: 2165-2178; Kozak et al., 2006 J Biol Chem 281: 29105-29119; Stopak et al., 2007 J Biol Chem 282: 3539-3546; Chelico et al., 2006 Nat Struct Mol Biol 13: 392-399; Sheehy et al., 2002 Nature 418, 646-650; Wichroski et al., 2006 PLoS Pathog 2: e41. Therefore experiments, were designed to target hA3G-RNA complexes to convert HMM to LMM in vivo. This offers as a novel therapeutic intervention for latent virus.

The examples presented therein demonstrate a method of assaying for agents that are useful for treating HIV invention. The examples presented herein relate to targeting hA3G:RNA complex as a strategy of dissociate hA3G and relieving it from RNA to allow for the antiviral activities of hA3G to defense against active or latent infection of HIV.

### Example 1

### Activation of Pre-Existing hA3G

The following experiments are based on the belief that that activators of APOBEC3G (A3G) can reduce the emergence of viral resistance. A3G has both enzymatic and nonenzyniatic properties that enable it to inhibit HIV replication (Holmes, et al., 2007, Trends Biochem Sci, 32:118-128). The efficacy of this host-defense factor is compromised in activated T cells due to its ability to bind nonspecifically to all forms of cellular RNAs and thereby oligomerize to form high molecular mass (HMM) aggregates (Chiu, et al., 2005, Nature, 435:108-114; Gallois-Montbrun, et al., 2007, J Virol, 81:2165-2178; Kozak, et al., 2006, J Biol Chem, 281:29105-29119; Kreisberg, et al., 2006, J Exp Med, 203:865-870; Stopak, et al., 2007, J Biol Chem, 282:3539-3546). The magnitude of RNA-dependent aggregation is such that it engulfs and inactivates virtually all of the A3G molecules in T cells following an HIV infection and inflammation. An additional compromise for host defense is that A3G does not immediately become reactivated as T cells enter the resting state (Santoni de Sio, et al., 2009, PLoS One, 4:e6571), suggesting that the emergence of viral resistance may in part be due to HMM and the absence of A3G antiviral activity within viral reservoirs. However there is controversy whether A3G that exists in cells can interact with incoming virus and inhibit their replication thereby making the cells nonpermissive or whether A3G must enter cells with viral particles in order to exert antiviral activity (Chiu, et al., 2008, Annu Rev Immunol, 26:317-353). Related to this is a debated over the importance of A3G interaction with host cell RNA or viral RNA for A3G encapsidation with virions (Strebel, et al., 2008, Retrovirology, 5:55). The high-risk aspect of the present invention is that a complete inhibition of A3G:RNA binding may inhibit its antiviral activity if encapsidation is the only means by which A3G can be antiviral. However the literature also shows that A3G encapsidation and its dC to dU deaminase activity on replicating viral DNA required that A3G not be bound to RNA as HMM (Chiu, et al., 2006, Trends Immunol, 27:291-297; Khan, et al., 2009, Retrovirology, 6:99; Opi, et al., 2006, J Virol, 80:4673-4682; Soros, et al., 2007, PLoS Pathog, 3:e15). Thus, it is proposed that A3G activators would reduce the tendency of A3G to form HMM aggregates and offer a major strategic advantage because they would enable host-defense during the early phase of the viral life cycle (a preemptive strike) prior to viral integration and before Vif-dependent A3G degradation and A3G encapsidation became important considerations.

Accordingly, experiments were designed to establish an assay for high throughput screening (HTS) for A3G activators (hits) based on in vitro assembled HMM complexes containing recombinant A3G and RNA. In addition, experiments were designed to assess whether the hits could be characterized as an antagonist but did not completely eliminate RNA binding to A3G.

As disclosed elsewhere herein, (i) expression of hA3G in quantities of >7 mg/ml with >90% of the material as LMM dimers or HMM tetramers depending on the inclusion of RNase has been accomplished, (ii) both LMM and HMM hA3G have been shown to bind exogenous RNA in vitro, (iii) gel shift analyses have shown efficient assembly of hA3G nucleic acid complexes with 24- and 41-mer probes, (iv) that while individual residues within the N-terminus of hA3G are necessary for binding to RNA, only full length hA3G actually binds RNA, (v) functional endpoints of in vitro deaminase activity and infectivity assays and (vi) consideration of commercial sources of qFRET donor-acceptor pairs and appropriate diversity set compound libraries for screening.

The following experiments were designed to activate pre-existing hA3G present in a cell by disrupting hA3G-RNA in HMM complexes. It is believed that disrupting hA3G-RNA complexes promotes antiviral activity for both deaminase-dependent and -independent mechanisms. Although HIV Vif from latent virus may continue to promote hA3G degradation, hA3G activation enables the cell to ‘strike back’ with antiviral activity.

An HTS assay was developed to screen for compounds that antagonize A3G binding to RNA and thereby reduce the RNA-dependent aggregation of A3G as HMM. The assay: (1) produces a positive signal for compounds that reduced the interaction of A3G with RNA, (2) has a good dynamic range between the assay background and the theoretical maximum signal and (3) was adapted for HTS in 384-well microtiter plate format. A quenched FRET (FqRET) assay has been established based on A3G:RNA complex formation that takes advantage of the ability of the compound QXL670 (coupled to RNA) to absorb and quench the fluorescence emitted form the compound Alexa647 (coupled to A3G) when irradiated by 647 nm light. Hits were identified through their ability to reverse the quench and induce red fluorescence at 670 nM.

### Example 2

### Validation of the Assay Components and their Interaction

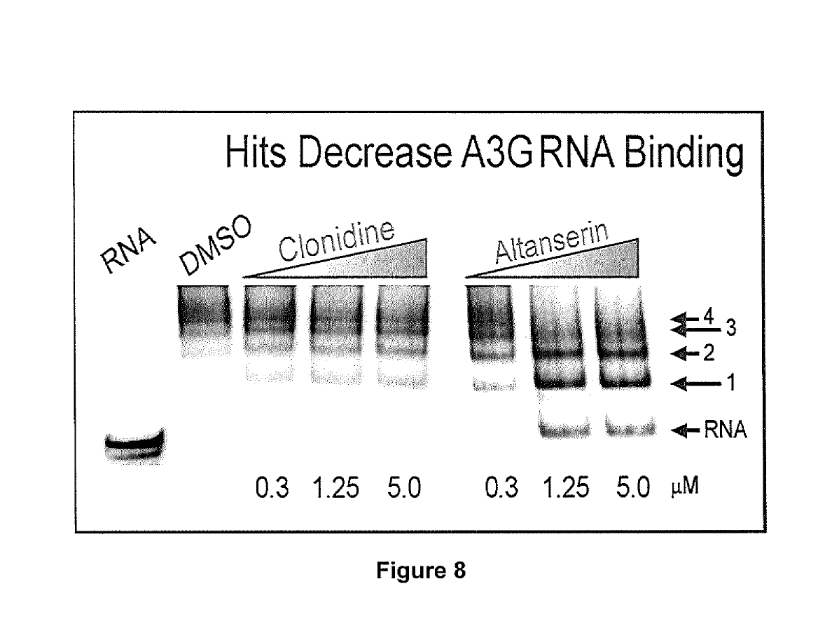

Experiments were performed to determine what length and sequence of nonspecific RNA would yield the most efficient formation of A3G:RNA complexes. A3G was expressed using the Baculovirus system and purified by nickel affinity chromatography. RNAs varying in GC and AU content were synthesized chemically or transcribed in vitro in lengths varying from 10 to 99 nucleotides (nt). A3G:RNA complexes were assembled in vitro with these RNAs over a range of A3G concentrations and the yield of small and large complexes determined by electrophoretic mobility shift on native gels (EMSA). These studies showed that GC-rich RNAs did not bind to A3G and AU-rich RNAs bound to A3G but with low affinity. An AU-rich RNA sequence containing an G or C every fourth nucleotide was identified as optimal for the studies as it had the highest binding affinity for A3G (Kd=30 nM) (an example of this RNA binding to A3G is shown in FIG. 8). The size of A3G HMM aggregates increased with increasing length of RNA while RNAs <20 nt failed to bind efficiently. A 99 nt RNA was selected for the HTS assay. The RNA used for the following data had the sequence:

(SEQ ID NO: 1)
GGGAACAAAAGCUGGGUACCGGGCCCCCCCUCGAGGUCGAUGCAGACAU
AUAUGAUACAAUUUGAUCAGUAUAUUAAAGAUAGUUAUGAUUUACAAG
CU

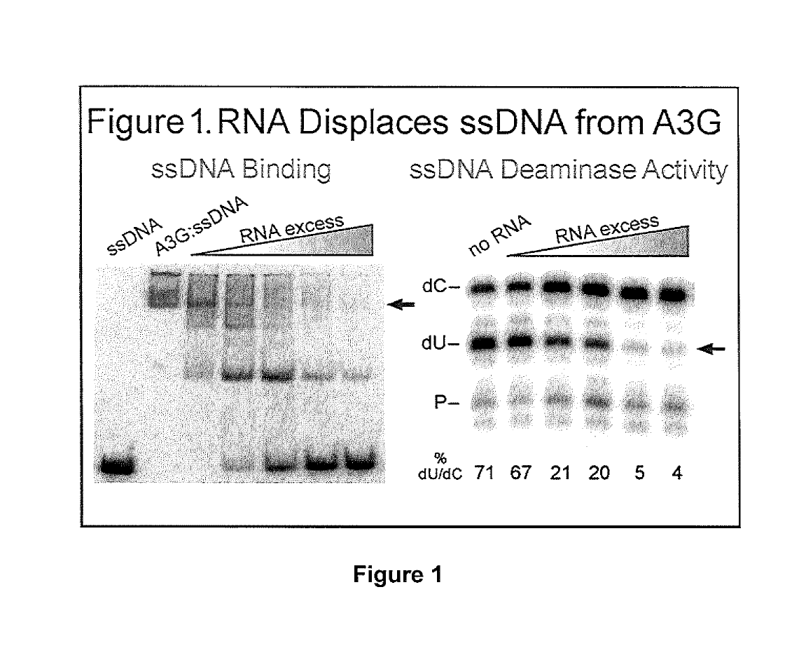

The next experiments were performed to determine the effect of RNA binding to A3G on A3G binding to DNA substrates. A3G:DNA complexes were assembled in vitro under standard deaminase assay conditions with radioactive DNA and analyzed by EMSA without or with prior competition from increasing concentrations of unlabeled 99 nt RNA (FIG. 1, left panel). The EMSA gel showed a fast migrating band of radiolabeled DNA without A3G (bottom left) that shifts to a slow complex with A3G is incubated with the DNA (second lane and indicated by the red arrow). When increasing amounts of unlabeled RNA was added to each assembly reaction from 0.5- to 100-fold molar excess of RNA:DNA, the A3G:DNA complexes were dissociated to smaller complexes with greater electrophoretic mobility and eventually DNA was liberated from A3G all together. The data demonstrated that RNA competes with DNA for A3G binding and thereby suggested one explanation for why A3G:RNA aggregates are not effective in host defense.

The ssDNA used for the EMSA and the served as a ssDNA substrate for the deaminase activity is a 41 nt ssDNA with the sequence:

(SEQ ID NO: 2)
ATTATTATTATTATTATTATTCCCAAGGATTTATTTATTTA.

It was believed that RNA binding would inhibit A3G deaminase activity on DNA. Using the same experimental conditions described above, DNA substrates were purified after in vitro incubation with A3G with DNA without or with RNA competition. The percent of DNA substrates with C to U changes due to deamination by A3G were determined by a primer extension sequencing for dU through the inclusion of the chain terminating nucleotide ddATP instead of dATP (FIG. 1, right panel). The reaction products were resolved by denaturing gel electrophoresis. The radiolabeled primer (P) extended to produce a long product (C) on unmodified DNA and a short product (U) (due to a ddATP induced stop to primer extension) on DNAs where A3G catalyzed a C to U modification. The reaction condition without RNA competition resulted in 71% of the DNA with U transitions, Competitor RNA induced a marked inhibition of deaminase activity. By comparing the EMSA and deaminase data, it was apparent that RNA-dependent dissolution of the active DNA deaminase complex corresponded with the maximum loss of deaminase activity. This finding is novel and showed that RNA inhibited A3G deaminase activity by displacing DNA substrates from A3G. The data also showed that relevant biological properties of A3G can be modeled in vitro.

### Example 3

### A Positive Selection Screen for the Disruption of hA3G-RNA Complexes Using Quenched Förster Resonance Energy Transfer (qFRET)

The following experiments were designed based on the rationale that RNA bound to A3G inactivates deaminase activity on ssDNA and removal of RNA reactivates antiviral activity. Positive selection for compounds that disrupt RNA binding to A3G are based on qFRET. Coupled FRET pairs (FRET donor and acceptor) are evaluated for optimal overlapping spectra wherein the acceptor quenches fluorescence of the donor but itself does not fluoresce or alter the native hA3G structure. The FRET quencher QXL™520 satisfies the above criteria, (AnaSpec, Calif.). Purified EGFP-hA3G (as FRET donor, emission at 509 nm) can be reacted in vitro with QXL520-containing RNA oligonucleotides. QXL520 is placed at different positions within the RNA during synthesis to optimize proximity of the FRET pair in the hA3G-RNA complex. To evaluate that hA3G quenching by the RNA probes is reversible, EGFP fluorescence and in vitro deaminase activity are used as endpoints of appropriate protein fold and function following RNase digestion. hA3G-RNA complexes formed with RNA lacking QXL520 should not quench (a negative control).

Without wishing to be bound by any particular filmy, it is believed that a screen using a positive readout for compounds that disrupt hA3G-RNA complexes is superior to a screen with a negative signal for a ‘hit’. Appropriate quenchers in FRET are chosen based on the wavelength at which they demonstrate absorption maxima relative to the emission spectra of the fluorescent molecule, as well as chemical characteristics that make them compatible with the buffer conditions of the experiment and amenable for conjugation. The fluorescence emitted from EGFP following laser excitation are quenched (made dark) when a compound capable of absorbing the quantum of energy emitted at the wavelength of EGFP (509 nm) is positioned within a short distance (typically 10-10≈) and with appropriate dipole (orientation) (Cullen, 2006, J Virol. 80: 1067-76; Peng et al., 2006, J Exp Med. 203: 41-6).

Nanomolar amounts of hA3G, as soluble fluorescent protein with an N-terminal or C-terminal EGFP (Bennett et al., 2008 JBC 283(12):7320-7), is titrated with increasing amounts of RNA conjugated 5′, 3′ or internally with the quencher QXL520 to achieve RNA binding and quenching of fluorescence. RNAs of various lengths and sequence can be commercially synthesized or transcribed in vitro for assembly with hA3G. Quenching activity resulting from hA3G-RNA complex formation is monitored by time-resolved fluorimetry in standard deaminase reaction buffer conditions. Gel shift analysis can be used to monitor hA3G-RNA binding efficiency.

It is important to identify the qFRET donor-acceptor pair that enables the best quenching of fluorescence. RNA plus albumin serve as the maximum quenched (dark) control and EGFP-hA3G alone or with unlabeled RNA serve as the maximum unquenched (fluorescent) control. RNase digestion of the reactions liberate EGFP-hA3G and can demonstrate that quenching was due to binding of the labeled RNA. Although EGFP-QXL520 donor-acceptor pair are matched in spectral overlap and energetics for qFRET other combinations of donor/acceptor are commercially available and can be explored to achieve the maximal quenching. For example, hA3G can be chemically coupled with the quencher QXL570 (at different ends and different R groups) and Cy3 can be incorporated into RNA during synthesis to produce the fluorescent donor.

### Optimization of the Assay for HTS

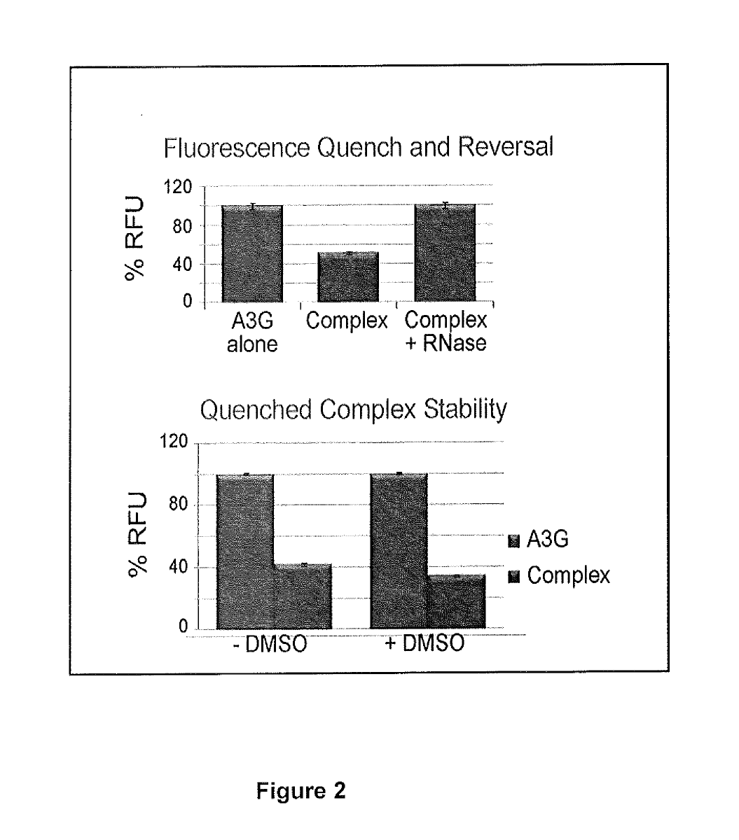

To establish the FqRET HTS assay, A3G was chemically coupled with Alexa Fluor647 and purified by size exclusion chromatography. The 99 nt RNA was transcribed in vitro with aminoallyl-UTP to introduce a site for chemical coupling with the quencher QXL670. RNA was incubated with QXL670 and QXL670-RNA was purified by gel electrophoresis. Alexa647-A3G:QXL670-RNA complex formation was verified by EMSA and these complexes demonstrated a >50% quenching of 670 nM fluorescence at an input of 1:5 A3G:RNA. The assay is ideal for HTS because it involves few robotic steps (a homogenous assay). Quenching was dependent on RNA binding to A3G as shown by the complete reversal of quenching upon RNase digestion of Alexa647-A3G:QXL670-RNA complexes (FIG. 2, top panel). As library compounds are dissolved in DMSO an important characteristic of the FqRET assay is that the fluorescent signal is not affected by up to 5% DMSO (FIG. 2, bottom panel). A standard in the HTS field for evaluating the ability of an assay to discern hit signals from background is the Z factor. Z is calculated as 1-[(3 sq+3 su)/(mean q−mean u)] where s=the standard deviation, q=quenched signal from Alexa647-A3G:QXL647-RNA complexes and u=the unquenched signal from Alexa647-A3G alone. An acceptable z factor would be 0.5. It was calculated that the Z factor for the assay in 384-well plates was 0.7 and therefore outstanding. Quenched A3G:RNA complexes were assembled in bulk manually and stored at −80° C. until they were dispersed robotically to 384-well plates. The complexes are stable to freezing and thawing. A range of concentrations of individual chemistries from libraries of drug-like small molecules were added to each well and four hours later the fluorescence from each well was quantified byrobotic plate reading relative to untreated (quenched) reactions as the baseline and Alexa647-A3G alone (as the maximum fluorescence). Alexa647-A3G, QXL670-RNA and Alexa647-A3G:QXL670-RNA complexes can be routinely produced in one day to screen 15,000 compounds.

The qFRET system can be optimize to a microtiter dish format for high through put screening. The conditions described elsewhere herein can be scaled to 384-well plate format and fluorescence quenching measured with a Perkin Elmer plate reader to calibrate the readout for screening. Without wishing to be bound by any particular theory, it is believed that screening chemical libraries requires high throughput such that the greatest number of compounds can be sampled in the shortest time. hA3G RNA binding conditions around the optima for quenching can be scaled down to the volume of 384, dark-wall microtiter dishes and analyzed using a Perkin Elmer plate reader.

The qFRET system can be used to screen compounds that disrupt hA3G-RNA complexes using a limited diversity set of small molecules. Non-limiting libraries that can be tested can be obtained from Life Chemistry, Maybridge, MyriaScreen, Sigma-Aldrich Screen that together, contain approximately >150,000 compounds in total; all conforming to Lipinski's rule of five and representative of a complementary but broad pharmacophore that has been used successfully to obtain ‘hits’ in screens for other HIV targets. Hits that reduce hA3G-RNA interactions and unblock deaminase activity are further evaluated for compounds that reduce infectivity but have no or low cell cytotoxicity.

Life Chemistry, Maybridge, MyriaScreen and Sigma-Aldrich libraries are frequently cited as appropriate diversity sets of drug-like compounds that broadly sample the pharmacophore. Compounds are tested across a range of 0.005 to 5 micromolar added during the assembly reaction. Current capacity enables set up and evaluation of thirty, 384 well plates/day. Accounting for wells with positive and negative controls, it is believed that ˜20,000 compounds can be evaluated a week. A ‘hit’ is scored as any compound with ≧50% enhancement of EGFP signal at any concentration. Hits are evaluated for potential side effects on ssDNA deaminase activity and viral encapsidation using dose-response analyses. Commercial available cytotoxicity assays (Promega) can be performed with each hit.

### Example 4

### Evaluating the Design of the Quenched Fret (FqRET) Assay

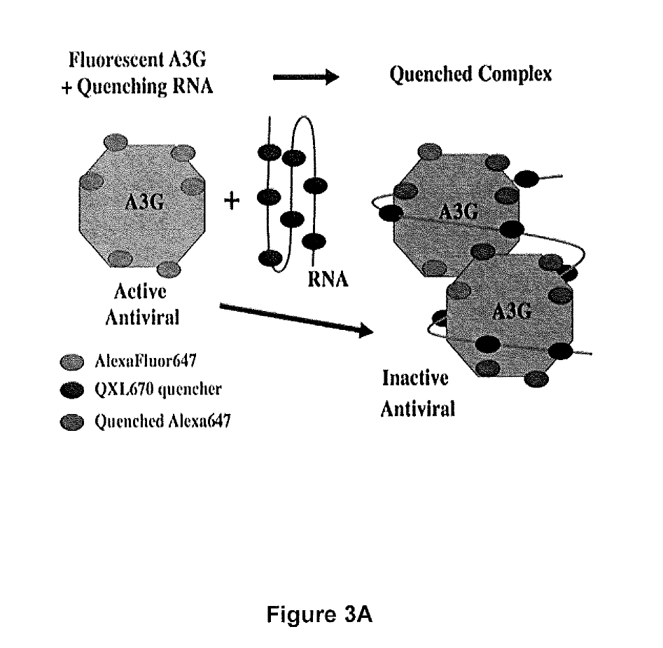

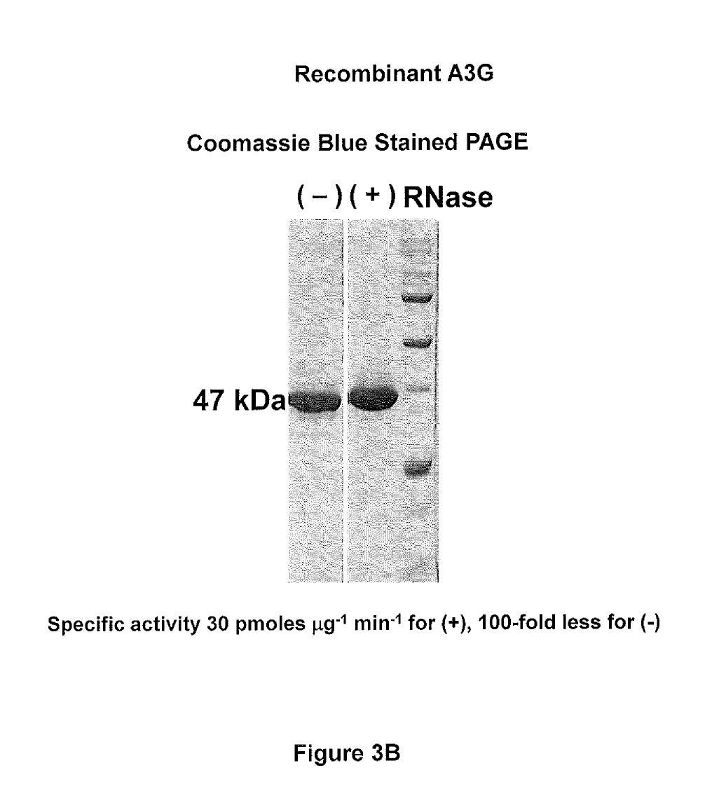

Experiments were designed to assemble fluorescently quenched, protein-RNA complexes consisting of recombinant APOBEC3G and RNA in the development of a high throughput screening (HTS) assay for compounds that disrupt A3G RNA binding and thereby activate the A3G enzymatic and antiviral activity (FIG. 3A). This assay enables selection of chemistries from a large compound library based on a positive signal as ‘hits’ impair or disable the ability of A3G to bind RNA and thereby relieve the fluorescence quenching. The development of the FqRET assay for A3G activators identified candidate activators of A3G host defense and have the potential to be first-in-class in HIV/AIDS therapeutics. The approach has been optimized based on three design considerations. (1) Several of the small molecule compounds that are in the libraries have green autofluorescence that could be misinterpreted as hits. False positive signals can be reduced in the assay by selecting a red fluorescence donor/acceptor pair. (2) A3G alone is readily expressed using Baculovirus-infected SD insect cells and can be purified as a soluble protein in multiple milligram quantities for structural studies. This recombinant A3G has been chemically coupled to the red fluorescent donor (Alexa647) that absorbs light at 647 nm and fluoresces at 670 nm (FIG. 3A). Consequently, the appropriate fluorescent acceptor (quencher) QXL670 has been coupled to RNA. (3) FqRET is a distance-dependent physicochemical phenomenon requiring close proximity of the donor and quencher. Given that there are no NMR or crystal structures for A3G-RNA complexes at this time, the amino acid residues within A3G that interact with RNA are unknown. However the noncatalytic domain in the N-terminus of A3G is known to be important for RNA binding (Opi, et al., 2006, J Virol 80:4673-4682; Navarro, et al., 2005, Virology 333:374-386) and the molecular envelope of A3G-RNA complexes determined by small angle X-ray scattering suggests that RNA is closely associated along the entire length of A3G (Wedekind, et al., 2006, J Biol Chem 281:38122-38126). Theoretically, quenching could result from RNA interactions along the entire surface of A3G. To maximize the efficiency of quenching, chemical coupling conditions were established such that Alexa647 and QXL670 could be coupled to multiple sites on A3G and RNA (respectively). Specifically, Alexa647 was purchased as N-hydroxy succinimidyl ester and reacted with purified A3G at a pH=7.2, a condition that activates all exposed primary and secondary amino groups of A3G for coupling to Alexa647. QXL670 also was purchased as an N-hydroxy succinimidyl ester and RNA was transcribed in vitro using a 1:1.5 molar ratio of aminoallyl UTP to UTP. Given that the RNA sequence contains 30% Us, the incorporation of aminoallyl UTP ensures that amino groups are available along the length of each RNA molecule for coupling to QXL670.

### Optimize A3G and RNA Chemical Coupling

15 to 20 mg of A3G can be purified from 2 liters of Baculovirus infected Sf9 insect cell culture. FIG. 3B shows Coomassie Blue stained gels of purified HMM and LMM (minus and plus RNase A digestion during protein purification). Coupling of Alexa647 to A3G has been optimized for reaction buffer conditions and temperature, duration of the coupling reaction and molar ratio of Alexa647 to A3G. Alexa647-A3G was purified by size exclusion chromatography with greater that 85% recovery of input A3G. In vitro transcription of the 99 nucleotide nonspecific RNA described previously for its high efficiency binding to A3G (Bennett, et al., 2008, J Biol Chem 283:33329-33336) was carried out with the mMessage mMachine kit from Ambion, Inc. Ten μg of aminoallyl labeled RNA was synthesized in each transcription reaction. QXL670 coupling was carried out in the transcription reaction and the QXL670-RNA was purified by polyacrylamide gel electrophoresis (PAGE). Recovery of QXL670-RNA from PAGE was ≧70%. The coupling reaction conditions could be varied over a broad range of protein, RNA or Alexa647/QXL670 input to produce A3G or RNA with different amounts of fluorescence and quenching. This flexibility is a strength as it enables optimizing of the assay's signal and detection limits.

### Establish Assay Conditions for A3G-RNA Complex Formation

It has been determined that chemically coupled A3G retained its ability to bind to RNA. The Electrophoretic Gel Mobility Shift Assay (EMSA) provides a visual and quantitative measure of the efficiency of A3G-RNA complex formation. EMSA was used initially to determine the optimum buffer conditions, molar ratio of A3G to RNA as well as the temperature and the duration of the complex assembly reaction. It was determined that the reaction conditions reported by Levin et al., (Opi, et al., 2006, J Virol 80:4673-4682) were efficient when carried out for 1 hour at 37° C. using a 2- to 5-fold molar excess of RNA to A3G.

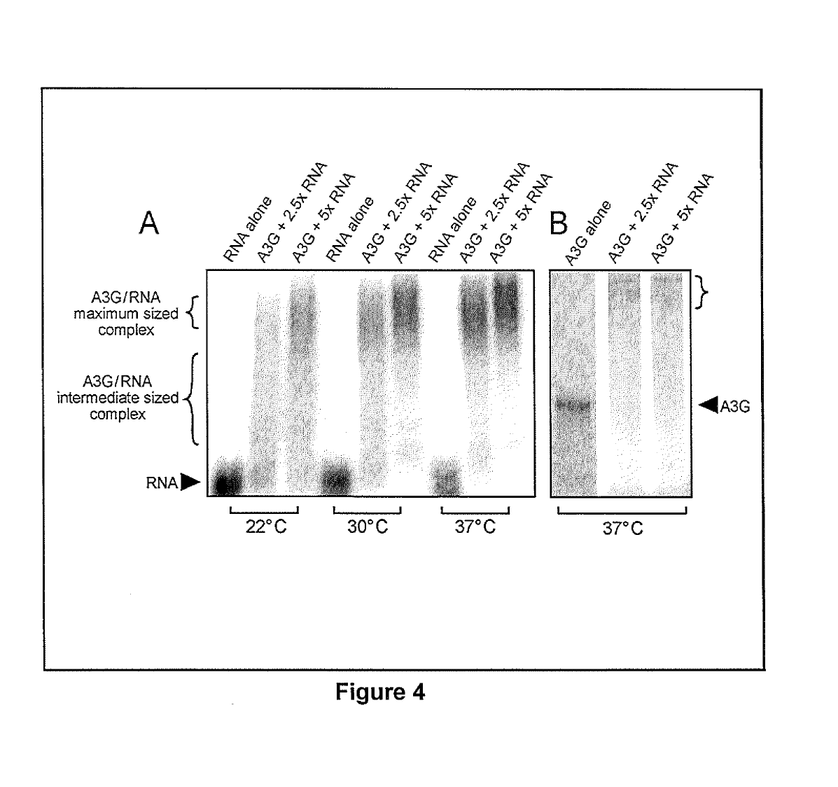

For these experiments, RNA was transcribed with aminoallyl UTP and α-³²P ATP to enable QXL670 coupling and radiographic visualization of gel shifted complexes. Each complex assembly reaction contained 0.01 nmols of A3G (0.5 μg) and the indicated molar excess of RNA. Electrophoretic mobility shift of ³²P-labeled RNA into larger A3G-RNA complexes demonstrated that chemically coupled A3G and RNA retained their ability to interact (FIG. 4A).

Intermediate sized complexes were apparent from reactions at lower temperatures but the recovery of maximum sized complexes was most efficient at 37° C. Aliquots from the 37° C. reactions as well as Alexa647-A3G from a reaction without QXL670-RNA were run on a second gel and scanned for fluorescence at 670 am following excitation at 647 nm. It was observed that Alexa647-A3G did not reacted with QXL670-RNA as measured by a fluorescent fast migrating band (FIG. 4B, A3G alone). Upon assembly with QXL670-RNA, the fluorescence of ALexa647-A3G was quenched, with much reduced fluorescence at the position where A3G-RNA complexes were anticipated to migrate based on the ³²P in FIG. 4A. In other EMSA experiments (not shown) it was determined that RNA-protein complex assembly was equally efficient when A3G was coupled with QXL670 and RNA was coupled to Alexa647. However, Alexa647-A3G and QXL670-RNA was elected as exemplary molecule to conduct further experiments.

Proof that Quenching was Due to the Formation of A3G-RNA Complexes

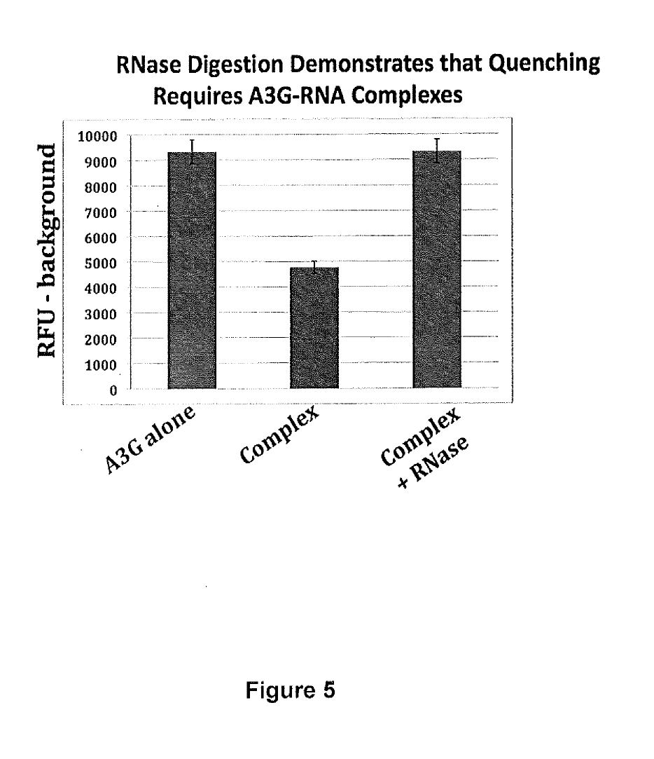

EMSA data provided strong evidence that A3G-RNA complexes had been assembled and that the chemistry coupled to the macromolecules and present in the complexes had the necessary physiochemical properties for FqRET. However, EMSA is not high throughput and there an exemplary high throughput screen includes the use of the FqRET system in the context of microtiter plates. To this end, A3G-RNA complexes were assembled in 30 μl reactions using a 1:5 molar ratio of donor to quencher. Quenching was determined to be as high as 55% in these reactions using a FluorMax-4 fluorometer equipped with a 43 μl cuvette (FIG. 5). Complete quenching was not anticipated given the generalized placement of donor and quencher long A3G and RNA. Importantly, the z factor for the assays was outstanding (0.7). RNase digestion removes RNA from A3G-RNA complexes and is known to reactivate A3G enzymatic ssDNA binding activities (Wedekind, et al., 2006, J Biol Chem 281:38122-38126; Soros, et al., 2007, PLoS Pathog 3:e15). It was observed that RNase digestion of the quenched complexes enhanced fluorescence (FIG. 5), demonstrating that A3G-RNA complex formation is integral to FqRET.

### Example 4

### High Throughput Screening (HTS)

The following experiments were designed to identify antiviral compounds for therapeutic development based on their ability to dissociate cellular RNAs from A3G and thereby activate host defense against HIV infectivity. An assay was developed based on A3G-RNA complexes assembled in vitro that would provide a positive signal upon dissociation of RNA from A3G. The assay is based on the biophysical technique of quenched FRET induced by the formation of A3G-RNA complexes. The assay has been adapted to the format necessary for high throughput screening (HTS) in 1536-well microtiter plates. The HTS assay is used to screen libraries of drug-like small molecules and evaluate the ‘hits’ for their ability to interact directly with A3G and inhibit HIV infection. Relevant compounds can be evaluated for their synthetic chemistry potential and predicted structure activity relationships (SAR). Subsequently, preliminary preclinical testing can be carried out in mice to determine T cell uptake and drug administration route, dosing, tissue metabolism, drug metabolism, metabolite excretion and toxicity (know collectively as ADMET).

Compounds that target A3G and activate its anti-HIV activity can be the precursors to first-in-class drugs. Unlike conventional therapeutics that target individual HIV proteins that are expressed at different stages of the HIV-1 infectivity cycle, A3G activation promotes host defense at early and late stages of viral infection. It is believed that activating A3G provide cells with a rigorous first line of defense against incoming HIV, disrupting the HIV genome as it replicates. It is also believed that activated A3G can escape Vif-dependent degradation by assembling with virions, unlike A3G-RNA complexes that do not become encapsidated and are degraded by Vif (Soros, et al., 2007, PLoS Pathog 3:e15). This places A3G in a physical proximity to strike down infecting HIV the moment the virus begins the next cycle of replication. Therefore, an additional attribute of activating A3G is reducing production of infectious virus from reservoirs even though Vif is expressed in these cells. These two mechanisms collectively may account for the correlation between elevated expression of A3G mRNA and reduced viral loads in PBMC of Long Term Nonprogressors (Jin, et al., 2005, J Virol 79:11513-11516). Without wishing to be bound by any particular theory, A3G activators of the presenting invention can be used in combination with Vif antagonists (compounds from traditional approaches) and part of HAART to reduce and/or eliminate viral resistance.

The HTS assay of the invention is useful for identifying a compound that has antiviral activity in the nanomolar range, binds to A3G with high affinity, has low toxicity and whose development to a lead compound through medicinal chemistry is predicted to be readily achieved.

A preliminary screen of a 20,000 compound ChemBridge Diversity Set library of drug-like small molecules can be carried out at the University's facility for HTS. Compounds in this library are guaranteed to be ≧95% pure. The screen can be conducted at a concentration of 1 μM for each compound. A hit is scored as a compound that produces ≧20% increase in fluorescence relative to the DMSO solvent treatment alone or the autofluorescence that may be due to the compound alone as it is believed that this is a realistic threshold for a true positive. A true hit however, demonstrates a dose response (where decreasing compound concentration results in proportionally less fluorescence) whereas a false hit (due largely to nonspecific effects of high chemical concentrations) ceases to produce a positive signal upon dilution. Initial hits are ‘picked’ from the library and reassembled as dilution series for re-screening. Hits are anticipated from the 20,000 compound library as it broadly represents chemical structures from across the known pharmacore and the industrial standard is that an assay with a z factor of ≧0.5 has a hit rate of 0.1% from such libraries.

The HTS assay of the invention can also be used to screen a ChemBridge Diversity Set library of 200,000 compounds. The importance of additional screening is that the larger library not only contains a broader diversity of chemical structures but importantly, also contains multiple variations of related chemical structures (analogs). The greater complexity in the library is anticipated to produce hits whose structures may bind with higher affinity to A3G (allosterically or directly) and dissociate RNA from A3G at lower concentrations. Compounds in this category are evident as increased fluorescence relative to background controls and at low concentrations of hits. Hits that bind to A3G but do not affect the ability of A3G to bind to RNA are not detected by the HTS assay. A hit from the larger library screen may be more representative of the chemical structure that might ultimately be developed as a lead compound.

Hits are assessed for their structural relatedness (cluster analysis) using computer assisted drug discovery (CADD) software. Hits can fall into a few structural classes (clusters) and it is anticipated that the library contains analogs within these classes that did not produce hits. All of this informative can be computationally analyzed by a desired commercial vendor who can evaluate the SAR to determine the best compounds to pursue and identify other analogs to test that were not in the original library but are generally available.

Compounds of interest are assembled into microtiter dish format by the commercial vendor and retested in the FqRET assay using the University of Rochester HTS facility as described elsewhere herein. The outcome enables refined computational SAR analysis through which <25 compounds can be selected for functional end-point analyses. Selection also can be biased for compounds whose availability and known synthetic and medicinal chemistry pathways are readily achievable. Compounds can be acquired and their structures and purity validated by mass spectroscopy at a commercial vendor and retested in a dilution series to determine the concentration of each compound that induces 50% and 95% increase in fluorescence relative to untreated controls.

Hits that inhibit HIV replication are initially determined using an assay based on pseudotyped Vif+ virus produced in A3G transfected 293T cells and the luciferase-based TZM-bl cell infectivity reporter assay (Platt, et al., 1998, J Virol 72:2855-2864). Compounds that reduce viral infectivity by ≧20% are evaluated over a range of doses to determine their IC₅₀ and IC₉₅. Compounds that demonstrate an IC₅₀ within the nanomolar to submicromolar concentration range are selected and sent to a commercial vendor to be evaluated for their antiviral efficacy against live HIV in human PBMC. These studies quantify infectivity based on p24 Gag immunological assays in 20 day spreading infection studies. Infected cells without compound treatment (negative control) and infected cells treated with AZT (positive drug control) are run as parallel analyses to assess the magnitude of each hit's antiviral activity. The dose-dependence of each compound can be determined. In a similar fashion, each compound's efficacy is evaluated against both R5 and X4 HIV strains (Cicala, et al., 2006, Proc Natl Acad Sci USA 103:3746-3751), different clades of HIV and strains of HIV with known drug resistant phenotypes. Relevant drugs to which the strains are known to be resistant or sensitive are evaluated in parallel assays as negative and positive infectivity controls, respectively. Suppression of the drug-resistant strains underscore the potential of A3G activators as novel therapeutics. A commercial vender also can evaluate antiviral activity of the hits in relevant cell types such as purified CD4+ T cells, resting memory T cells and in PHA/IL2 stimulated PBMC. It is anticipated that ≦6 hits can be identified for further functional end-point analysis based on their dose-dependent antiviral activity in the relevant cell types. These compounds can be evaluated for three functional end-points.

(i) Dissociating nonspecific cellular RNAs from A3G is believed to increase the amount of A3G that is incorporated into viral particles. This is apparent as an increase in A3G western blot signal when viral particles purified from compound-treated infected cells are compared with particles isolated from infected cells treated with DMSO alone. These data can be quantified by Phosphorimager scanning densitometry using the p24 western blot signals as a normalization control for viral particle input as described previously (Bennett, et al., 2008, J Biol Chem 283:33329-33336).

(ii) Activation of A3G is believed to reduce viral replication and induce hypermutation of the proviral genome. DNA extracted from pseudovirus-infected cells that have or have not been treated with compound can be quantified by real time PCR to determine the extent to which hits reduce proviral DNA expression using HIV specific amplimers. The viral DNA sequences can be evaluated for dG to dA hypermutations with an A3G nearest neighbor signature (Yu, et al., 2004, Nat Struct Mal Biol 11:435-442; Hache, et al., 2005, J Biol Chem 280:10920-10924) through the sequencing service of the University's genomic center.

(iii) To identify compounds that bind directly to A3G, recombinant A3G prepared free of RNA can be immobilized through its C-terminal polyhistidine tag to BiaCore chips designed for nickel affinity binding and analyzed on a BiaCore X instrument. Polarized light incident on the surface of chips containing A3G can be reflected and detected as the signal. The angle of the reflected light changes when A3G binds to compounds and changes conformation and the change in reflected light angle (surface plasmon resonance, SPR) is measured relative to light reflected from A3G without compound added. SPR has a broad linear range up to 70,000 RU and a sensitivity of 100 RU (where 1 RU=1 pg of bound compound per mm²). Relevant hits can be evaluated for A3G binding over a broad range of concentrations. From the resultant RU, association (Ka) and dissociation (Kd) constants for each compound can be calculated using software packages available with the BiaCore X. SPR is essentially a mass detector and errors in measurement increase as the size of the macromolecule increases. The ability of compounds to dissociate A3G-RNA complexes therefore can be carried out using isothermal calorimetery or ITC that does not only enable confirmation of the BiaCore quantification but also uniquely enable the determination of thermodynamic parameters associated with changes in A3G-RNA interactions. The biophysical parameters enable advanced SAR and quantitative metrics for medicinal chemistry.

It is believed that a number of hits are identified as having significant potential for lead compound development based on functional endpoint analysis, high level of efficacy in the nanomolar concentration range and interaction with the A3G-RNA target. An important foundation at this stage is to establish the toxicity of the compounds. Hits are tested first over an appropriate range of doses (based on the infectivity studies) for cell toxicity in human umbilical vein endothelial cells using the ‘Biological Profiling/Counter Screening’ services at the HTS center of Yale University. These studies can reveal whether compounds affect cell viability, morphology, cell cycle progression or induce stress response.

The compound(s) with low toxicity can be administered over a broad range of doses to mice using the facilities and services of a commercial lab services. Studies can be designed for the number of animals needed to achieve statistical significance. Animal administration and toxicology can begin with 5 each of age-matched males and females as sham treated animals and 5 male and female animals for each dose of compound tested, Weight gain, behavioral and metabolic assessments can be conducted. Treatments can be considered to have low general tissue toxicity if individual alterations in daily food consumption and weekly whole body or organ weight are within the observed normal range expected for untreated animals+/−15%. T-cell uptake of each compound can be determined. ADMET can be planned based on the experimental design created by a skilled artisan. An appropriate commercial vender can advise for the medicinal chemistry to produce compounds with reduced toxicity and to improve uptake and distribution.

### Example 5

### Selection of Hits on A3G:RNA Complexes by HTS

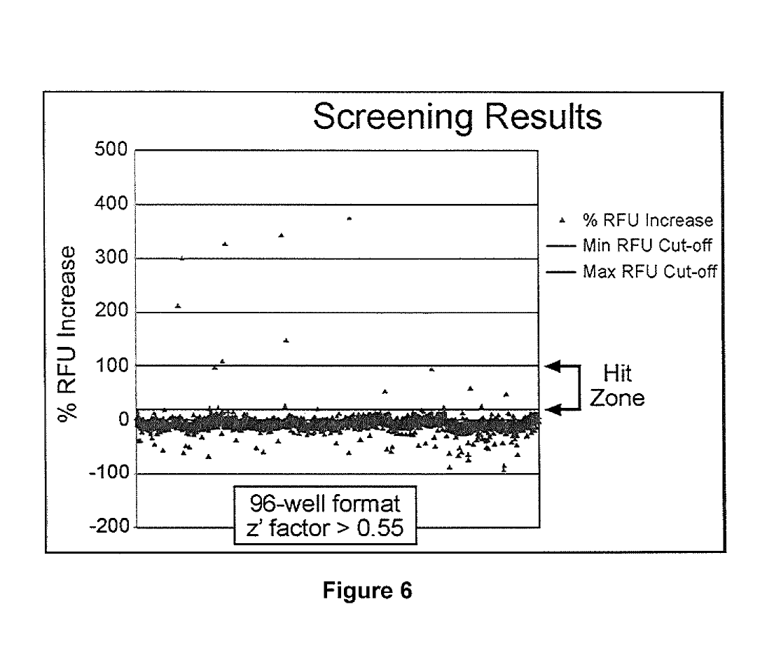

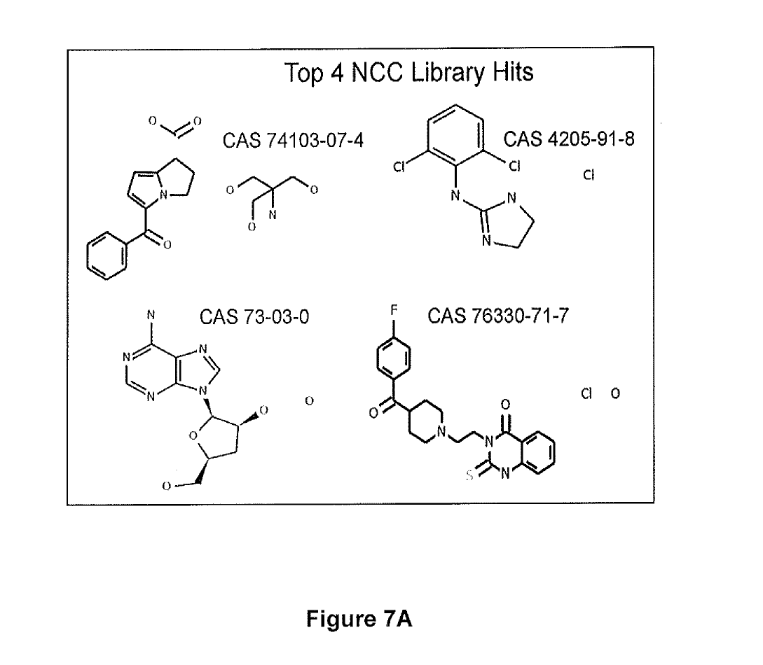

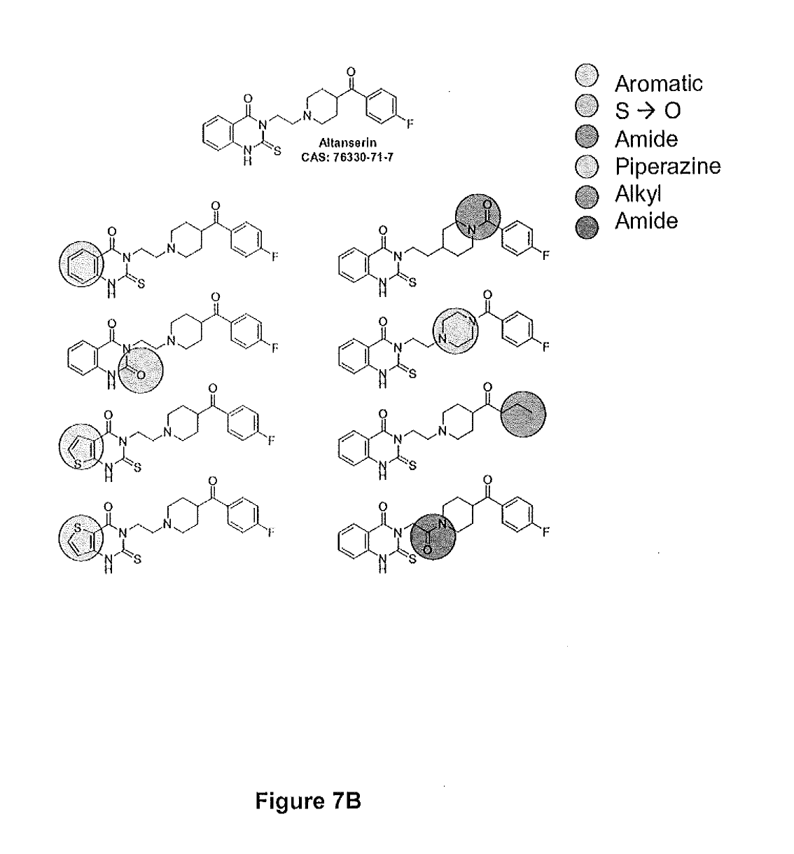

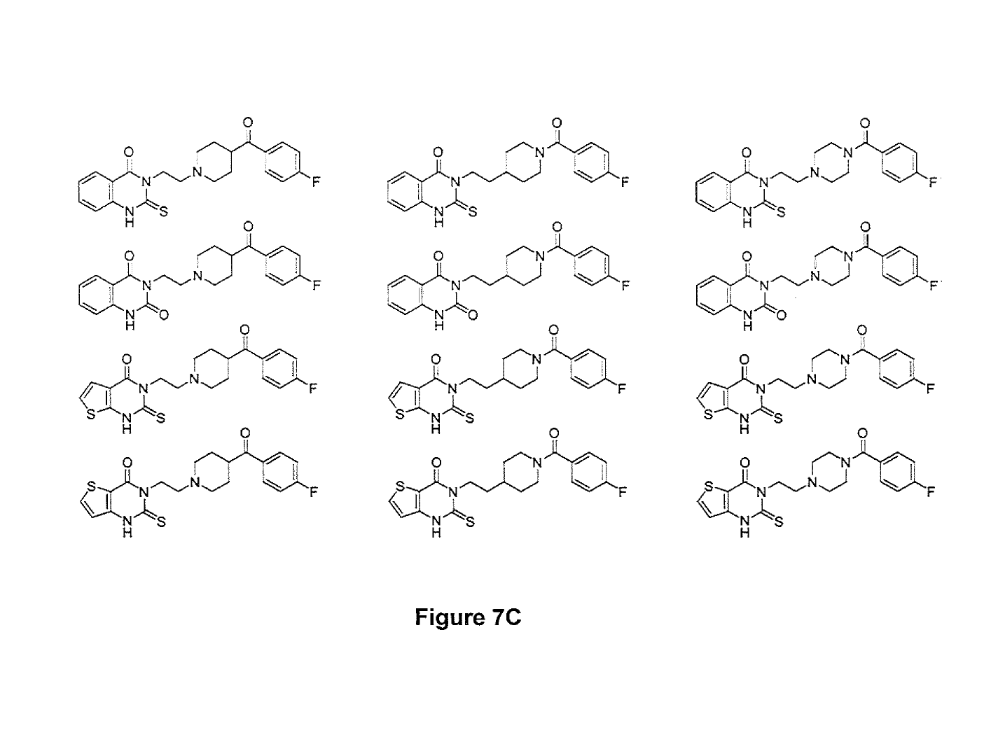

Ideally, a robust HTS should have a low background and be able to discriminate false positives that result from autofluorescent compounds. The number of compounds identified per total number of compounds screened is the ‘hit rate’ which for good assays is 0.1% to 0.5%. A HTS assay in 96-well format using the 2,000 compound Spectrum library and 446 compound National Clinical Collection library has been developed. These libraries consisted of diverse off-patent drugs together with a small diversity-set of drug-like compounds. Each library compound was initially screened at 20 uM. Graphic representation of the analysis (FIG. 6) showed only eight compounds induced fluorescence within the ‘hit zone’ between the quenched baseline and the maximum anticipated fluorescence for unquenched Alexa647-A3G. This corresponded to a hit rate of 0.32%. Compounds in the library that were autofluorescent and emitted above the anticipated maximum fluorescence was not pursued. Four compounds were selected for further study (FIG. 7) because they showed a dose-dependent ability to induce fluorescence between 1 uM to 20 uM.

### Example 6

### Validation of Hits for RNA Binding Antagonists

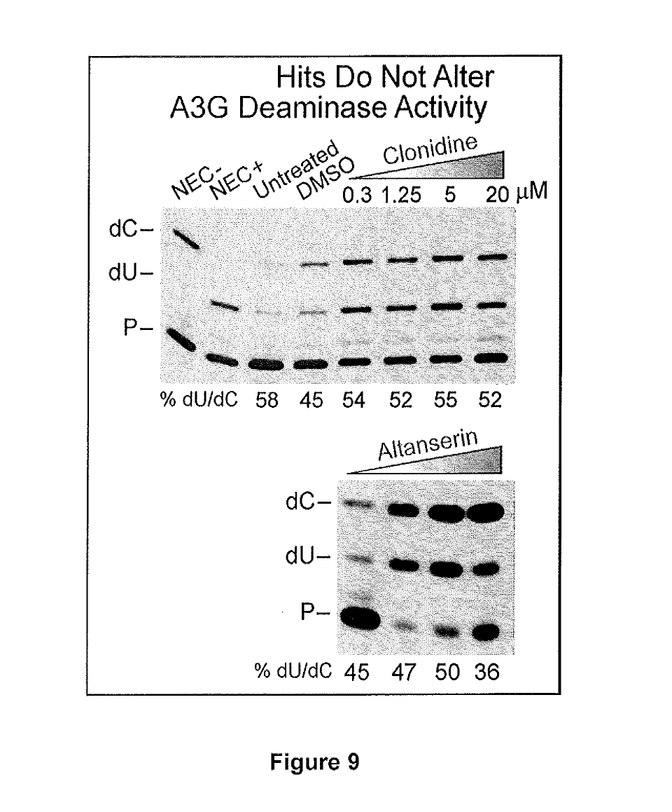

The four compounds depicted in FIG. 7 were evaluated for their ability to disaggregate A3G:RNA complexes by treating A3G:RNA complexes assembled on radiolabeled RNA with each chemistry over a range of concentrations and then evaluated for size reduction of HMM by EMSA. DMSO (or compound that were not hits in the original screen) that did not affect the size of slow electrophoretic mobility HMM (FIG. 8). All four hits demonstrated to varying degrees a dose-dependent dissolution of A3G:RNA aggregates to faster migrating lower molecular mass (LMM) complexes, exemplified by Clonidine (CAS 74103-07-4). Altanserin (CAS 76330-71-1) was the only hit that had the ability to completely dissociate RNA from a portion of the A3G complexes, as evidenced by the appearance of free RNA in addition to LMM complexes at the highest Altanserin concentrations. None of the hits induced RNA degradation. The compounds had their maximum effect when they were preincubated with A3G prior to the addition of RNA, suggesting that they were binding to A3G and inhibiting the ability of A3G to form RNA-dependent aggregates. Purified A3G was treated with each of the hits at varying concentrations and subsequently assayed for DNA deaminase activity as described elsewhere herein, Remarkably, none of the hits inhibited A3G deaminase activity (exemplified by clonidine and Altanserin, FIG. 9). The hits were not toxic to the cells up to 50 uM when assayed using a commercially available Cell Death Assay (Cell TiterGlo, Promega). The data demonstrated antagonists of A3G:RNA complex formation and importantly the hits partially disaggregated HMM but did not inhibit A3G DNA deaminase activity.

### Example 7

### Antiviral Activity of the Hits

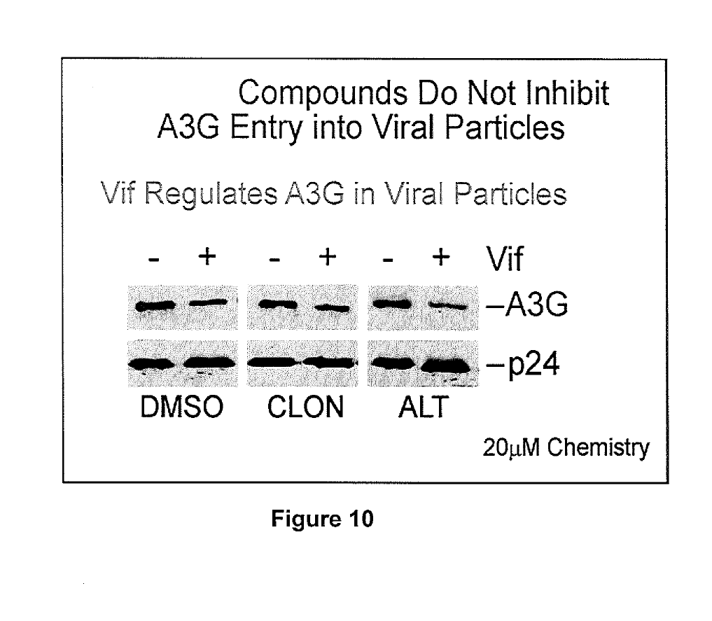

The antiviral activity of each hit was evaluated using two different single round infectivity assays each based on pseudotyped virus produced in the HEK 293T cells with or without the stable expression of A3G in order to address the concern of whether the compounds would inhibit A3G packaging with virions. Viral particles were produced in HEK 293T cells that had stable A3G expression by transfecting them with proviral HIV DNA that was minus env and either did or did not express functional Vif and co-transfected with the VSV env gene. Five hours after transfection 20 μM of either chemistry was added or DMSO as a control, Pseudotyped viruses were harvested 24 hours post-transfection from each condition. Upon normalization of the viral preparations by p24 ELISA, a western blot analysis on equivalent nanogram quantities of each virus was performed to evaluate viral particle incorporation of A3G (FIG. 10). The data demonstrated that amount of A3G incorporated into viral particles was not affected by the addition of chemistry but as expected, Vif expression had a dominant effect in reducing the recovery of A3G in the viral particles. Viral particles with more A3G (minus Vif) were less infective as anticipated by the literature.

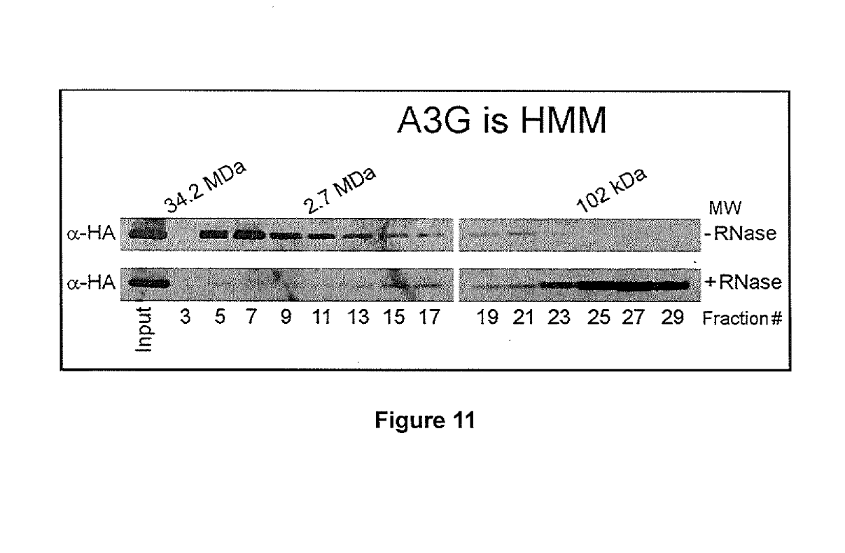

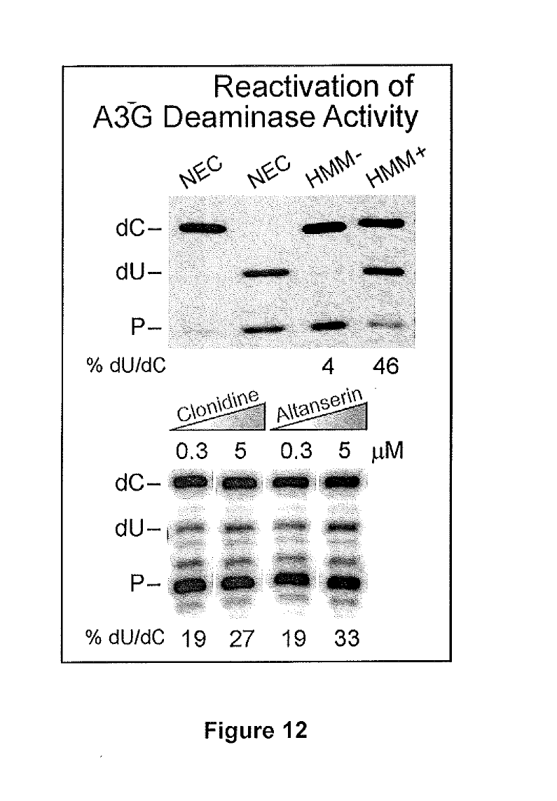

Experiments were performed to directly test whether the compounds could activate antiviral activity from HMM in living cells. For this a cell line that expressed A3G as HMM was created. TZM-bl reporter cells were transfected with A3G cDNA and a cell line with stable expression of A3G was selected (TZM-bl-A3G). To verify HMM A3G, cell extracts were prepared from TZM-bl-A3G cells and fractionated by size exclusion chromatography. Western blots of the fractions using anti-A3G antibodies showed that A3G was aggregated as MDa, RNase-sensitive HMM (FIG. 11). Isolated HMM had low levels of in vitro deaminase activity (4% dC to dU) that could be activated 10-fold by RNase digestion (FIG. 12, top panel). Treatment of HMM with 20 μM Clonidine and Altanserin activated A3G deaminase activity 6- and 8-fold respectively compared to untreated HMM (FIG. 12, bottom panel). The data demonstrated that the selected hits target A3G:RNA complexes assembled in cells.

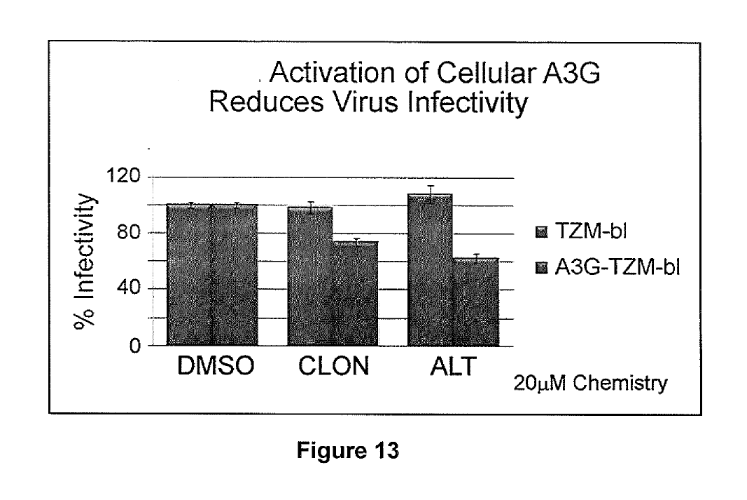

To evaluate the antiviral activity of the compounds in cells, pseudotyped viruses with or without a functional Vif gene were produced in HEK 293T cells that did not express A3G. Viral particles were harvested from media of producer cells, normalized based on p24 content and equal number of virus particles were used to infect wild type TZM-bl or TZM-bl-A3G cells. As anticipated from the literature (Chelico, et al., 2006, Nat Struct Mol Biol, 13:392-399), HMM had no antiviral activity as indicated by the infectivity of viruses +/− Vif in both cell lines treated with DMSO alone (FIG. 13). However, HIV replication and, consequently Tat expression, are anticipated to be inhibited if dissolution of HMM by A3G activators was able to stimulate host defense. Consistent with the proposed mechanism of action of A3G activators, Altanserin, and to a lesser extent Clonidine induced antiviral activity in TZM-bl-A3G cells but not in TZM-bl cells (FIG. 13). Altanserin inhibited >40% of the viral infectivity at a single dose of 20 uM. The antiviral activity of these compounds was not affected by the presence of a functional Vif gene, a predictable outcome as Vif would have only been expressed in the late viral life cycle.

Altanserin is a known 5-HT_{2A} receptor (serotonin 2A receptor) antagonist and has been approved for use in humans for PET imaging of 5-HT_{2A} receptor expression in the central nervous system and is well tolerated. Clonidine interacts with α₂-receptors in the brain and is used to treat hypertension. FDA approval for both compounds would streamline repurposing for HIV/AIDS clinical trials. However, the chemical framework of these compounds affords a unique opportunity for medicinal chemistry structure activity relationship (SAR) studies and the identification of a chemotype with optimized target selectivity and low nanomolar antiviral efficacy.

### Example 8

### Drugs and Delivery Systems to Limit Drug Resistance

The emergence of drug-resistant strains of HIV is due in part to the high mutation frequency of reverse transcriptase during viral replication and viral genetic recombination that are ongoing at levels below immune covalence in viral reservoirs. The antiviral activity of A3G arises from its ability to physically block progression of the viral replication machinery as well as to bind to nascent proviral DNA and catalyze multiple mutations through dC to dU transitions (deamination). These activities are absent when activated T cells return to their resting state (Santoni de Sio, et al., 2009, PLoS One, 4:e6571) because A3G remains sequestered in high molecular mass (HMM) aggregates. HMM complexes may be composed of multiple (4 to >20) inactivated A3G subunits tethered together through nonspecific binding of A3G to cellular RNAs (Chin, et al., 2005, Nature, 435:108-114; Gallois-Montbrun, et al., 2007, J Virol, 81:2165-2178; Kozak, et al., 2006, J Biol Chem, 281:29105-29119; Stopak, et al., 2007, J Biol Chem, 282:3539-3546; Chelico, et al., 2006, Nat Struct Mol Biol, 13:392-399; Sheehy, et al., 2002, Nature, 418:646-650; Wichroski, at al., 2006. PLoS Pathog 2:e41).

The present invention is based on the discovery that selectively targeting A3G binding to RNA and HMM formation to activate host defense can be used as an anti-viral therapy. It has been observed that specific amino acids in the N-terminal, pseudocatalytic domain of A3G (Sheehy, et al., 2002, Nature, 418:646-650; Huthoff, et al., 2009, PLoS Pathog, 5:e1000330; Iwatani, et al., 2006, J Virol, 80:5992-6002; Navarro, et al., 2005, Virology, 333:374-386; Bennett, et al., 2008, J Biol Chem, 283:33329-33336; Shandilya, et al., 2010, Structure, 18:28-38; Wedekind, et al., 2006, J Biol Chem, 281:38122-38126) were involved in RNA-dependent A3G aggregation. In contrast, DNA was bound, and dC's deaminated through a catalytic domain at the C-terminus of A3G (Sheehy, et al., 2002, Nature, 418:646-650; Iwatani, et al., 2006, J Virol, 80:5992-6002; Navarro, at al., 2005, Virology, 333:374-386; Shandilya, at al., 2010, Structure, 18:28-38). The C-terminal half of A3G in isolation was sufficient for DNA binding and deamination whereas RNA binding to A3G and inhibition of deaminase activity required full length A3G (Bennett, at al., 2008, J Biol Chem, 283:33329-33336).

The disclosure presented herein demonstrate that RNA binding to A3G inhibited deaminase activity by inducing the enzyme to release its DNA substrate. RNA binding to the N-terminus of A3G is believed to induce a protein conformational change that disfavored DNA binding at the C-terminus (Navarro, et al., 2005, Virology, 333:374-386) or RNA is believed to competed directly for DNA binding at the C-terminus.

Prior to the present invention, traditional thinking has held that A3G must be encapsidated to be antiviral and that inhibiting Vif was the only way to enable A3G host defense. For example, recent RNAi knockdown experiments have challenged the importance of A3G antiviral activity by showing that the reduction of A3G expression in nonpermissive cells was not sufficient to make cells permissive to HIV infection (Santoni de Sio et al., 2009 PLoS One 4: e6571, Kamata et al., 2009 PLoS Pathog 5: e1000342). These findings supported an earlier study of normal and HIV infected patients that suggested A3G expression levels did not correlate with viral load (Cho et al., 2006 J Virol 80: 2069-2072). A reasonable conclusion from these studies is that A3G is not the sole cellular defense mechanism against HIV infection. Arguing in favor of a significant role of A3G in host defense has been the discovery of compounds through HTS that maintained cellular levels of A3G when Vif was co-expressed (Nathans et al., 2008 Nat Biotechnol 26; 1187-1192. These compounds enabled A3G to assemble with viral particles and reduced HIV infectivity. The studies supported an earlier report that long term nonprogressing patients (LTNP) had a higher expression level of A3G than uninfected controls or patients with HIV/AIDS (Jin et al., 2005 J Virol 79: 11513-11516; Vazquez-Perez et al., 2009 Retrovirology 6: 23, Ulenga et al., 2008 J Infect Dis 198: 486-492). An interesting corollary was that viral genomes isolated from LTNP contained a high proportion of mutations in the Vif gene (Janini et al., 2001 J Virol 75: 7973-7986. The controversy in the field stems from the fact that current research reagents cannot address the question of whether activation of A3G in permissive cells will make them nonpermissive. In this regard, compounds of the invention that target A3G:RNA complexes have already been identified and shown to be strategically important in addressing this question. For example, the present invention provides an unconventional solution to the important problem of viral resistance in that the invention provides a way to overcome HIV resistance to host defense mechanisms by activating A3G with compounds that dissociate A3G-RNA complexes.

The results presented herein demonstrate an assay for understanding of RNA-protein interactions and identification of agents that exhibit novel antiviral properties by being able to disrupt RNA-protein interactions such as A3G-RNA complexes. It has been demonstrated that: (i) A3G DNA deaminase activity was stimulated by compounds that antagonized A3G binding to RNA and HMM formation and (ii) viral replication was inhibited when permissive cells expressing A3G as HMM were treated with A3G-activating compounds. This is a high level of success that could not have been anticipated from the literature because traditional thinking is that A3G must be encapsidated to be antiviral and that inhibiting Vif is the only way to enable A3G host-defense.

The next set of experiments were designed to test whether RNA inactivation of A3G as MINI is reversible and once A3G is activated whether it exerts antiviral activity against incoming virus. Without wishing to be bound by any particular theory, it is believed that A3G activators antagonize nonspecific binding of RNA to A3G, inhibit viral replication and integration and therefore not depend exclusively on A3G encapsidation for therapeutic efficacy.

Accordingly, aspects of the invention are based on the unpredictable nature of the finding that RNA binding to A3G is reversible in vitro and in living cells. This finding is unpredictable particularly based on the fact that the art was understood that A3G needed to be in the particle to have an antiviral effect. In the contrary, the present invention is based on the discovery that A3G can preemptively attack incoming virus and does not have to be in the virus to be antiviral. Thus proving that A3G is an important antiviral and for the first time addressing the controversy of whether more A3G is a better defense against HIV.

It is believed that one or more novel antiviral compounds exhibit with nanomolar efficacy and low toxicity whose mechanism of action is validated as being through the novel target. It is believed that activation of A3G reduces viral infectivity and the emergence of viral resistance by empowering the host with an additional means of ‘fighting back’. The present invention offers the ability to protect cells from HIV through a post entry inhibition of viral replication.

### Conduct Structure-Activity Relationship (SAR) Analysis of the Chemical Scaffold and R-Groups Based on the Identification of Altanserin and Clonidine

Eighty compounds have been identified that are both variations of the core scaffold of clonidine and altanserin and have varying R-groups and these compounds are synthesized for Structure Activity Relationship (SAR) studies. The compounds are rescreened through the FqRET HTS assay discussed elsewhere herein to select chemistries reactive with the target.

Hits are evaluated over a range of doses to identify compounds with the highest therapeutic value based on four functional endpoints: (i) the lowest IC50 and IC95 as determined in single round viral infectivity assays, (ii) the highest recovery of A3G with viral particles, (iii) the ability to dissociate A3G:RNA complexes based on EMSA (iv) while having low or no effect on in vitro deaminase activity.

Compounds are re-evaluated in a secondary FqRET assay for A3G binding to nonspecific RNA versus HIV RNA or 7SL RNA to identify compounds that markedly enhance A3G encapsidation.

Based on these studies, the appropriate compounds can be selected for additional SAR analysis that includes the design and testing of modifications of these compounds to: (i) reduce their IC50/IC95 and (ii) reduce or eliminate their toxicity.

Quantities of A3G and RNA suitable for structural studies that validate drug-target interactions can be readily produced. The University of Rochester's structural biology core equipment and services for surface plasmon resonance (BiaCore) and isothermal calorimetry (ITC) can be used to determine: (i) the affinity of compounds for A3G, (ii) the compound on and off rate kinetics for A3G binding and (iii) quantify RNA and DNA binding to A3G over a range of compound concentrations.

Hits are evaluated for their ability to block viral replication using qPCR to quantifying proviral DNA and replication intermediates in treated or untreated infected cells. The mutation frequency is quantified in PCR amplified proviral genomes of compound-treated infections relative to untreated controls (+/−A3G expression) to assess the mutation frequency due to activation of A3G deaminase activity as part of the antiviral mechanism.

Conduct HTS with the FqRET Assay Using a Larger Compound Library

The development of therapeutics based on a novel and innovative target requires a comprehensive understanding of the chemotypes reactive with the target and their ability to modify the activity of the target. The identification of compounds from, for example, off-patent compounds that have antiviral activity and reactivity with the novel target of the invention is a significant advance, indicating that there are likely to be other compounds with desirable characteristics. This potential can only be realized by HTS of libraries with greater chemical diversity.

The FqRET assay is used to screen a diversity set library of drug-like small molecules, for example, commercially available from ChemBridge. Hit identification, hit validation and SAR analysis are performed as discussed elsewhere herein. Validated hits are subjected to chemical cluster analysis and SAR analysis as discussed elsewhere herein.

### Live Virus Infectivity Testing

Single round infectivity assays provide a good first evaluation of the antiviral activity of compounds and a conventional assay that is based on VSV envelope pseudotyped HIV to evaluate each compound's antiviral efficacy during viral particle production can be used. A single round infectivity assay can be used which is based on pseudotyped virus produced in HEK 293T cells (embryonic kidney cell line) and infectivity of luciferase reporter expressing HeLa cells (cervical carcinoma cells). While this is an effective first test that is broadly used in academia and industry to evaluate HIV infectivity, recent studies have shown that the VSV env protein may change the dynamics of the virus-host cell interactions (Yu, et al., 2009, PLoS Pathog, 5:e1000633). To ensure the greatest likelihood of success in clinical trials, it is believed that it is important to determine the efficacy of the GCE compounds through testing with live HIV virus and human white blood cells (PBMC).

Compounds, for example those that have come through medicinal chemistry, are tested over a range of drug doses (50 μM to 0.5 nM) in 21-day spreading infections using human PBMC infected with live HIV-1NL4-3 at initial viral inputs varying from of 0.01 to 1.0 moi. The IC50 and IC95 are determined using a HTS reverse transcriptase activity from cell lysates as a measure of viral infectivity. The relative antiviral efficacy of the GCE compounds are assessed by comparing their ability to reduce HIV burst phase and spreading infection compared to that seen for infected but untreated cells and infected cells treated with a conventional RT inhibitor as an antiviral positive control.

Compounds are evaluated for their antiviral efficacy against virus in 21-day spreading infection using virus derived from different geographical regions (clades). Compounds are evaluated for their antiviral efficacy on 3 different multidrug resistant strains. Multiple rounds of spreading infection can be conducted with sub-effective low dose of relevant GCE compounds and the resulting virus can be tested against for the emergence of a drug-resistant strain over a range of doses of GCE compounds.

### Animal Toxicology Testing

As a precondition to FDA approval new anti HIV/AIDS therapeutics must be evaluated in two different species for route of administration, dosing, metabolism, excretion and toxicology (ADMET) and because of their DNA mutagenic activity, A3G activators can be evaluated for genotoxicity by quantifying the changes in the occurrence of single nucleotide polymorphisms (SNPS) in the genome of treated animals compared to sham controls using ‘deep’ sequencing technology. ADMET is conducted in mice using a commercial lab services. Whole animal evaluations as well as metabolic and blood chemistry endpoints determine (i) the maximum tolerated single dose, (ii) plasma drug concentration following single and multiple dosing regimen, (iii) compound half life and (iv) metabolism and excretion.

The next set of experiments is to assess preclinical ADMET and efficacy using a nonhuman primate species and SIV. Efficacy testing prior to human Phase I/IIa clinical trails is possible because cellular RNA-dependent aggregation and inactivation of A3G occurs in all mammals.

The disclosures of each and every patent, patent application, and publication cited herein are hereby incorporated herein by reference in their entirety.

While the invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

What is claimed:

1. A method of identifying an agent that disrupts A3G:nucleic acid molecule interaction, said method comprising contacting A3G in an A3G:nucleic acid molecule complex with a test agent under conditions that are effective for A3G:nucleic acid molecule complex formation, and detecting whether or not the test agent disrupts A3G:nucleic acid molecule interaction, wherein detection of disruption of A3G:nucleic acid molecule interaction identifies an agent that disrupts A3G:RNA nucleic acid molecule.

2. The method of claim 1, wherein said nucleic acid molecule is selected from the group consisting of ssDNA, RNA, and any combination thereof.

3. The method of claim 2, wherein the test agent that disrupts A3G:RNA interaction activates its ssDNA dC to dU deaminase activity as part of an inhibitor of lentiviral infectivity.

4. The method of claim 2, wherein the test agent that disrupts A3G:RNA interaction enables binding to ssDNA in lentiviral replications complexes as part of an inhibitor of lentiviral infectivity.

5. The method of claim 1, wherein said method is a high throughput method.

6. The method of claim 1, wherein said high throughput method is Förster quenched resonance energy transfer (FqRET).

7. An agent identified by the method of claim 1.

8. A method for inhibiting infectivity of a virus, the method comprising contacting a cell with an antiviral-effective amount of an agent identified by the method of claim 1.

9. The method of claim 8, wherein the virus is selected from the group consisting of HIV 1, HIV 2, hepatitis A, hepatitis B, hepatitis C, XMRV, and any combination thereof.

10. The method of claim 8, wherein the virus is associated with an RNA intermediate in the cytoplasm of cells.

11. The method of claim 8, wherein the virus is associated with DNA replication in the cytoplasm of cells.

12. The method of claim 8, wherein the virus comprises endogenous retroviral elements of the line, sine, and alu category.

13. The method of claim 8, wherein the virus is a foamy virus.

14. The method of claim 8, wherein the agent inhibits the interaction of A3G with RNA, thereby allowing the A3G to exhibit anti-viral activity.

15. The method of claim 8, wherein said agent is selected from the group consisting of Altanserin, Clonidine, and analogs thereof and having a related chemical scaffold (chemotype).

16. A method for inhibiting A3G:RNA interaction in a cell, said method comprising contacting A3G:RNA complex with an inhibitory-effective amount of an agent identified by the method of claim 1.

17. The method of claim 16, wherein said agent is selected from the group consisting of Altanserin, Clonidine, and analogs thereof and having a related chemical scaffold (chemotype).

18. A method for treating or preventing HIV infection or AIDS in a patient, the method comprising administering to a patient in need of such treatment or prevention a therapeutically effective amount of an agent identified by the method of claim 1.

19. The method of claim 18, wherein said agent is selected from the group consisting of Altanserin, Clonidine, and analogs thereof and having a related chemical scaffold (chemotype).

20. A method of attacking viral resistance, the method comprising releasing RNA inactivation of A3G thereby activating A3G in a cell.

21. The method of claim 20, wherein A3G is not encapsidated in order to exert its antiviral activity.

22. The method of claim 20, wherein the cell has not been infected by a virus and activation of A3G t preemptively inhibits viral replication.

23. The method of claim 20, wherein releasing RNA inactivation of A3G is accomplished by contacting a cell with an antiviral-effective amount of an agent identified by the method of claim 1.

24. The method of claim 20, wherein releasing RNA inactivation of A3G is accomplished by contacting a cell with an antiviral-effective amount of an agent selected from the group consisting of Altanserin, Clonidine, and analogs thereof and having a related chemical scaffold (chemotype).

25. A method of creating a reservoir of an active form of A3G in a cell prior to viral infection of the cell, the method comprising disrupting A3G:RNA complex in the cell.

26. A method of reducing the emergence of viral drug-resistance in a cell, the method comprising disrupting A3G:RNA complex in the cell.


SEQUENCE LISTING (2 sequences)

SEQ ID NO: 1 - RNA, 99 nt, Homo sapiens
gggaacaaaagcuggguaccgggcccccccucgaggucgaugcagacauauaugauacaauuugaucaguauauuaaagauaguuaugauuuacaagcu

SEQ ID NO: 2 - DNA, 41 bp, Artificial sequence
  Chemicially synthesized
attattattattattattattcccaaggatttatttattta